(19) United States
(12) Patent Application Publication
(10) Pub. No.: US 2023/0363889 A1
(43) Pub. Date: Nov. 16, 2023

(54) ACCOMODATING INTRAOCULAR OPTIC ASSEMBLIES

(71) Applicant: JITANDER DUDEE, LEXINGTON, KY (US)

(72) Inventor: JITANDER DUDEE, LEXINGTON, KY (US)

(21) Appl. No.: 18/197,452

(22) Filed: May 15, 2023

Related U.S. Application Data
(60) Provisional application No. 63/341,433, filed on May 13, 2022.

(51) Int. Cl. A61F 2/16

(52) U.S. Cl. CPC A61F 2/1629; A61F 2210/0085; A61F 2210/0061; A61B 3/16

(57) ABSTRACT

Improvements to accommodating intraocular optic assemblies are disclosed herein. The accommodating intraocular optic assembly can include an optic and at least one stanchion. The at least one stanchion can extend a length between a base end and a distal end. The distal end can be operably engaged with the optic directly or indirectly. The at least one stanchion can include an outer sleeve defining a through-aperture. The at least one stanchion can also include at least one inner member positioned within the through-aperture.

### CROSS-REFERENCE TO RELATED APPLICATIONS

This application claims the priority benefit of U.S. Provisional Patent Application Ser. No. 63/341,433 for Enhanced Stanchions for Accommodating Intra-Ocular Lenses (IOLs), filed on May 13, 2022, which is hereby incorporated by reference in its entirety.

### BACKGROUND

### 1. Field

The present disclosure relates to structures positionable in a human eye such as intraocular lens arrangements, drug delivery systems, sensor holders, and glaucoma treatment devices.

### 2. Description of Related Prior Art

Prosthetic intraocular lenses (IOLs) are routinely implanted following cataract extraction in human eyes and have grown in sophistication in order to provide better functional visual acuity with fewer troublesome distortions, reflections and aberrations to images focused on the retina. However, the natural lens retains distinct advantages over currently available IOLs. One such quality is the ability to alter its optical power to allow clear focusing on near as well as distant objects through human volition in tandem with contraction of the ciliary muscle of the eye. The physiological mechanism whereby the human eye voluntarily alters its focal point from distance to near is termed “near-accommodation” and a prosthetic lens implant, or “optic,” that seeks to perform this function is termed an Accommodating IOL or AIOL, or an accommodating intraocular optic assembly. Several designs have been proposed in the prior art for AIOLS that attempt to achieve the variable focus distance of the youthful natural lens but all have significant limitations.

U.S. Pat. Pub. No. 2005/0027354 discloses a PRIMARY AND SUPPLEMENTAL INTRAOCULAR LENS. The intraocular lens system includes a primary intraocular lens configured to correct vision in a patient, and a supplemental intraocular lens configured to modify the correction provided by the primary intraocular lens. The supplemental intraocular lens, which is substantially completely diffractive, is preferably ultrathin. The two lenses may be connected to, or separate from, one another. The supplemental intraocular lens may be implanted at the same time as the primary intraocular lens, or added later.

U.S. Pat. Pub. No. 2008/0288066 discloses a TORIC SULCUS LENS. There is disclosed therein a “piggyback” cylindrical (toric) intraocular lens for placement in front of an accommodating or standard intraocular lens that is already in the capsular bag of the eye. This additional lens is placed in the sulcus, which leaves a significant space between the two lenses, particularly if the lens in the capsular bag is vaulted backwards.

U.S. Pat. No. 8,425,597 discloses ACCOMMODATING INTRAOCULAR LENSES. Intraocular lenses for providing accommodation include an anterior optic, a posterior optic, and a lens structure. In one such lens, the lens structure comprises an anterior element coupled to the anterior optic and a posterior element coupled to the posterior optic. The anterior and posterior elements are coupled to one another at a peripheral region of the intraocular lens. The intraocular lens may also include a projection extending anteriorly from the posterior element that limits posterior motion of the anterior optic so as to maintain a minimum separation between anterior optic and an anterior surface of the posterior optic.

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Improvements to accommodating intraocular optic assemblies are disclosed herein. The accommodating intraocular optic assembly can include an optic and at least one stanchion. The at least one stanchion can extend a length between a base end and a distal end. The distal end can be operably engaged with the optic directly or indirectly. The at least one stanchion can include an outer sleeve defining a through-aperture. The at least one stanchion can also include at least one inner member positioned within the through-aperture.

Improvements to accommodating intraocular optic assemblies disclosed herein further include material selection for the at least one stanchion to increase stiffness in response to body temperature or through hydration.

Improvements to accommodating intraocular optic assemblies disclosed herein further include a pressure sensor assembly configured to detect pressure within the eye.

### BRIEF DESCRIPTION OF THE DRAWINGS

The detailed description set forth below references the following drawings:

FIG. 1 is a cross-sectional side view of a first exemplary accommodating intraocular optic assembly in a first exemplary operating environment in which features disclosed herein can be utilized;

FIG. 2 is a front view of the first exemplary accommodating intraocular optic assembly;

FIG. 3 is a cross-sectional side view of a portion of second exemplary accommodating intraocular optic assembly in a second exemplary operating environment in which features disclosed herein can be utilized;

FIG. 4 is a perspective view of the second exemplary accommodating intraocular optic assembly;

FIG. 5 is a cross-sectional side view of a portion of third exemplary accommodating intraocular optic assembly in a third exemplary operating environment in which features disclosed herein can be utilized;

FIG. 6 is a cross-sectional side view of a portion of fourth exemplary accommodating intraocular optic assembly in a fourth exemplary operating environment in which features disclosed herein can be utilized;

FIG. 7 is a cross-sectional side view of a portion of fifth exemplary accommodating intraocular optic assembly in a fifth exemplary operating environment in which features disclosed herein can be utilized;

FIG. 8 is a cross-sectional side view of a portion of sixth exemplary accommodating intraocular optic assembly in a sixth exemplary operating environment in which features disclosed herein can be utilized;

FIG. 9 is a side view of a first embodiment of an improvement in accommodating intraocular optic assemblies in the form of a stanchion configured to provide selective, post implant stiffening;

FIG. 10 is a detail view of a second embodiment of an improvement in accommodating intraocular optic assemblies in the form of a portion of a stanchion configured to provide selective, post implant stiffening;

FIGS. 11A and 11B are perspective views of a third embodiment of an improvement in accommodating intraocular optic assemblies in the form of a stanchion configured to provide selective, post implant stiffening;

FIGS. 12A, 12B, and 12C are views of a fourth embodiment of an improvement in accommodating intraocular optic assemblies in the form of a portion of a stanchion configured to provide selective, post implant stiffening;

FIGS. 13A and 13B are side views of a fifth embodiment of an improvement in accommodating intraocular optic assemblies in the form of a stanchion configured to provide selective, post implant stiffening;

FIGS. 14A and 14B are side views of a sixth embodiment of an improvement in accommodating intraocular optic assemblies in the form of a stanchion configured to provide selective, post implant stiffening;

FIGS. 15A and 15B are side views of a seventh embodiment of an improvement in accommodating intraocular optic assemblies in the form of a stanchion configured to provide selective, post implant stiffening;

FIGS. 16A and 16B are side views of an eighth embodiment of an improvement in accommodating intraocular optic assemblies in the form of a portion of a stanchion configured to provide selective, post implant stiffening;

FIG. 17 is a schematic view of a first embodiment of an improvement in accommodating intraocular optic assemblies in the form of an intraocular pressure sensor; and

FIG. 18 is a front view of a second embodiment of an improvement in accommodating intraocular optic assemblies in the form of an intraocular pressure sensor.

### DETAILED DESCRIPTION

The present disclosure, as demonstrated by the exemplary embodiments described below, provides a plurality of improvements to accommodating intraocular optic assemblies in which an optic such as lens or a ring member is mounted within an eye and held in place by a plurality of stanchions.

A plurality of different embodiments of the present disclosure is shown in the Figures of the application. Similar features are shown in the various embodiments of the present disclosure. Generally, similar features across different embodiments have been numbered with a common reference numeral and have been differentiated by an alphabetic suffix. Also, generally, similar features in a particular embodiment have been numbered with a common two-digit, base reference numeral and have been differentiated by a different leading numeral. Also, to enhance consistency, the structures in any particular drawing share the same alphabetic suffix even if a particular feature is shown in less than all embodiments. Similar features are structured similarly, operate similarly, and/or have the same function unless otherwise indicated by the drawings or this specification. Furthermore, particular features of one embodiment can replace corresponding features in another embodiment or can supplement other embodiments unless otherwise indicated by the drawings or this specification.

The following terms are useful in the defining the operating environment of one or more embodiments of the present disclosure:
Intraocular Lens or “IOL” refers to a prosthetic optical lens placed within the eye to allow better visual functioning of the eye;
“Conventional IOL” refers to an IOL that has a single fixed focal point (also known as a monofocal IOL);
“Near Accommodation” or “Accommodation” refers to a change in the focal point of the optical system of the human eye from fixation on distant objects (those further away than about 6 meters from the eye) to near objects (those closer than about 0.5 meters from the eye), the term “accommodation” also includes the act of focusing on objects in the intermediate range of 6 to 0.5;
“Ciliary Body” or “CB” refers to the Ciliary Body of the eye including the various neuromuscular elements comprising the structure commonly referred to as the Ciliary Muscle, as well as the connective tissue joining the muscular elements and forming attachments of the ciliary muscle to the sclera and to the zonules or suspensory ligaments of the lens capsule. The muscular tissue within the CB is generally of the type known as “smooth muscle”. Many microscopic muscle cells are connected to each other via elastic connective tissue forming bundles or rings of muscle that contract and stretch as a result of the combined contraction of the constituent muscle fibers;
“Ciliary Body accommodation” or “CBA” refers to the anatomical and physiological changes initiated by the act of voluntary human accommodation, during CB accommodation, impulses from the brain are transmitted to the nerves supplying the ocular tissues so that at least one eye is directed to align its optic axis towards the object of visual fixation, when at least one eye fixates on an object of visual interest, subconscious cues create an approximate estimate of the distance of the object from the eye and CB accommodation is triggered to the appropriate approximate extent required for the image from the object to be sharply focused on the retina, a process of reiterative biofeedback occurs so that the degree of CB accommodation is matched to the required working distance for sharp focus of the image from the object that is being viewed, other physiological actions are also linked to CB accommodation such as convergence (inwards rotation of eyes to triangulate and focus on a near object) and miosis (constriction of pupils to increase visual depth of field);
“Lenticular accommodation” refers to the alteration in optical power of the youthful or pre-presbyopic human eye in response to CB accommodation, the natural human lens is also known as the crystalline lens. It is enclosed within the lens capsule which in turn is connected to the ciliary body via many zonules (also known as suspensory ligaments) that attach close to the peripheral equator of the lens capsule on its posterior and anterior surfaces and extend in a radial fashion, suspending the crystalline lens from the CB. CB accommodation results in increased relative curvature of the front and rear lens capsule surfaces (also known collectively as the capsular bag), and a forward shift in the optical center of the crystalline lens, lenticular accommodation occurs as a result of decreased radial tension in the zonules because CB accommodation causes a relative anterior shift of the ring formed by the center of radial suspension the zonules, the cross sectional diameter of the eyeball is less at the relatively anterior location of the CB ring during CB accommodation, therefore the tension in the zonules is decreased allowing the elastic crystalline lens to revert to a shape that is more rounded in its anterior and posterior curvatures;
“Ciliary Sulcus” Refers to the ring like space bounded posteriorly by the ciliary process and suspensory ligaments of the lens (zonules) and bounded anteriorly by the posterior surface of the iris, the ciliary sulcus is bounded peripherally by the soft tissues overlying the ciliary body, these soft tissues separate the ciliary sulcus from the muscular components of the ciliary body, specifically the circular or annular portions of the ciliary muscle, the meridional portions of the ciliary muscle lie more peripherally and are anchored at the scleral spur, the ciliary sulcus extends for 360 degrees at the base of the iris, is vertically oval in humans and decreases in diameter during CBA;
“UBM” or “Ultrasound biomicroscopy” refers to imaging studies of the eye which show characteristic biometric changes that occur during ciliary body contraction, for understanding of the intended working of embodiments of this present disclosure, it is necessary to define some biometric features that change during CBA:
SSD (sulcus-to-sulcus diameter)—distance between opposite points in the ciliary sulcus, this will vary between individuals due to normal anatomic differences depending on the axial location of the opposite points because the ciliary sulcus is oval instead of circular in the near accommodated state in comparison to the relaxed state as CBA reduces SSD,
ICPA (Iris-ciliary process angle)—the angle between the plane of the iris and the direction of the ciliary process from between which the lens zonules extend to the equator of the capsular bag,
ACA (anterior chamber angle)—the angle between the plane of the peripheral iris and the inner layer of the cornea where they meet close to the iris root;
“Annular muscle contraction” or “AMC” refers to the morphological changes occurring during the contraction and relaxation of an annular or sphincteric muscle, specifically, it relates to the shape changes of the round portion of the ciliary muscle during CBA, the ring shaped “round” portion of the ciliary muscle encloses a central opening known as a lumen, which forms the external boundary of the ciliary sulcus, when an annular muscle contracts its total volume remains essentially unchanged but the circle surrounding the lumen in the plane of the lumen constricts, each point lining the lumen moves in relation to its neighbor during contraction and relaxation so that there are no two points that remain stationary relative to each other;
“Elastic biological surface” or “EBS” refers to a flexible membrane that forms the outside enclosure of an annular muscle or other elastic biological surface such as the capsule (or capsular bag) of the crystalline lens;
“Point-to-point contraction linking” or “PPCL” refers to the ability of a device to remain in contact with an elastic biological surface during the entire cycle of contraction and expansion without slipping at its contact points and without offering sufficient resistance to impede movement or cause damage by abrasion or penetration, for a device to be usefully coupled to an annular muscle (such as that found in the CB) it is essential for the device to offer in a predictable manner only as much resistance to movement as is necessary to convert the contraction of the muscle (in this case the contraction associated with CBA) into useful work (in this case IOL accommodation or “IOLA”), effective PPCL depends on critical design elements related to the points of contact of the device to the elastic biological surface, the features in point of contact design to achieve effective PPCL include:
distribution and location—Points of contact should be located around a center of movement that is also the center of movement of the elastic biological surface,
number—The points of contact should be numerous enough to maintain stable attachment during motion and distribute resistance evenly across biological surface, at least eight contact points can be desirable for PPCL to a device within the lumen of an annular muscle, too many points of contact if large will limit movement by causing crowding and if small, may impede biological function by causing scarring,
size—large contact points in contact with elastic biological surfaces such as the ciliary sulcus or capsular bag will present resistance against contraction or expansion of those surfaces, the continuous expansion and contraction of an annular muscle (even with its surrounding connective tissue) against an inelastic surface is likely to cause damage to biological tissues by abrasion and deposition of eroded tissues, contact points that are too small are likely to cause damage by perforation or penetration into biological tissue,
profile—curved contact points offer a variable surface area and some degree of “rocking” during expansion and contraction which protects biological tissue and reduces scarring, multiple protrusions are vulnerable to becoming entangled during implantation, becoming damaged or causing damage to biological tissue;
“Haptic Vaulting” when used in relation to IOLs refers to forward or backward movement of IOL optic in the direction of the visual axis relative to the distal ends of its haptics, in prior art Haptic vaulting is envisioned as a mechanism for achieving IOLA in capsular bag fixated IOLs in response to decreasing diameter of the capsular bag which may vertically compress the haptic ends, Haptic Vaulting may occur surreptitiously in even prior art conventional or monofocal IOLs, depending on nature and placement of the haptics within a fibrosed or contracted capsular bag;
“Rigid Vaulting” when used in relation to IOLs refers to forward or backward movement of IOL optic in the direction of the visual axis relative to the optical nodal point of the eye in response to mechanical forces within the eye, specifically, this relates to movement of an IOL fixed within a capsular bag (IOL-capsule diaphragm) in response to movements of the entire capsular bag caused by:
contraction or relaxation of the zonules attached to the capsular bag secondary to ciliary muscle contraction,
variations in fluid pressure (from aqueous humor or vitreous humor) between the anterior and posterior surfaces of the IOL-capsule diaphragm,
gravitational shifting of IOL in response to changes in eye position (Rigid Vaulting is widely believed to occur surreptitiously in prior art conventional or monofocal IOLs, but to a variable and unpredictable extent and therefore cannot be relied on to provide useful degree of IOLA);
“Pseudo-accommodation” refers to the retention of some functional unaided near vision in combination with good distance vision following cataract extraction in patients who do not have IOLA, in patients who have a fixed focal length IOL implanted, whose power is set for clear distant vision, it is the ability of such patients to have better than expected (although still limited)near vision (without reading glasses), its existence is due to the following factors or fortuitous conditions:
Pinhole effect—increased depth of field caused by decreasing aperture of the pupil during CBA and in conditions of high illumination, this effect may be enhanced in some lenses whose central curvature is higher than peripheral so that when the peripheral cornea is curtained off by the constricting pupil, the overall focus of the lens because closer, relying on the pinhole effect has the disadvantage of reducing amount of light available to the eye and hence compromising the overall quality of vision,
Aspheric optic property of the IOL (Lens has more than one major focal point). This may be intentional or serendipitous: Multifocal IOL design including pupil independent (diffractive lenses, aspheric curvatures) and pupil assisted (linked to pupillary constriction like the pinhole effect but accentuated by the IOL deliberately having a higher power in its central curvature, and Fortuitous/serendipitous optical effects presenting a secondary near image due to lens tilt (induced lenticular astigmatism) and corneal myopic astigmatism (Asymmetry of corneal curvature or tilting of the IOL can cause astigmatism, for example in which vertical lines far away, are seen better than horizontal lines, with the reverse holding try for near, since writing tends be composed of vertical and horizontal lines, people with just the right degree of astigmatism learn to decode the otherwise blurred near vision), and Limited accommodation due to IOL forward movement during CBA which may occur with any IOL implanted in elastic capsular bag with intact zonular attachments where the IOL-capsular bag complex moves forward during CBA increasing the effective power of the IOL and causing its focal point to move from distance to near, younger post cataract patients are often seen to have less need for reading glasses than expected when their (non-accommodating) IOLs have been selected for distant focus in both eyes, it is thought that the combination of a vigorous scarring response (causing the posterior capsule to bind firmly around the edge of the lens, and still strong ciliary muscles, allows the IOL to move forward in a way similar to the natural lens, this effect is usually not of sufficient extent to obviate the need for reading glasses;
“Monovision” refers to the illusion of good near and far vision obtained by implanting a monofocal IOL in one eye whose focal point is for distance and another monofocal IOL in the fellow eye whose focal point is for near. Monovision can also achieve a form of pseudo-accommodation so that when both eyes are used together, one provides good monocular distance vision and the other provides acceptable monocular near vision if the brain is able to adapt to this method of correction, this technique is often not well tolerated and causes reduction in stereoscopic vision, the patient is able to use each eye for its working distance (distance or near) although this does not represent true accommodation;
“IOL accommodation” or “IOLA” refers to a change in the optical focal point of an intraocular lens (hereafter IOL) from a sharp distant focus to a sharp near focus (and intermediate distances when the object of visual attention is in between) in an attempt to simulate is lenticular accommodation in response to CB accommodation, IOL accommodation is not equivalent to the IOL multifocality achieved by multifocal IOLs described immediately below;
“Multifocal IOL” or “MFIOL” refers to an IOL designed to have multiple simultaneous focal point, MFIOLs offer a degree of pseudo accommodation by having multiple focal powers or curvatures molded into a single IOL resulting in images of objects at more than one working distance becoming focused simultaneously on the retina, however, the simultaneous presentation of more than one image by the IOL causes degradation and compromise of each of the images as well as troublesome visual symptoms of halos, glare, ghost images collectively known as dysphotopsia, the providential persistence of pupillary miosis associated with CB accommodation can be utilized to preferentially select the central portion of the IOL curvature for near focusing and allow input from the peripheral lens curvature when CB accommodation is relaxed, and the pupil becomes relatively dilated, however, this type of “pinhole effect” also compromises overall quality of the images and multifocal IOLs in general have limited utility because CB accommodation does not result in true IOL accommodation, the increased range of focus depth of field presented by a static multifocal IOL is offset by lower image quality and visual aberrations, the eye and brain have to learn to ignore the images that are not useful for the current working distance and therefore there is compromise in overall vision quality and comfort;
“Haptic” refers to an arrangement of structural elements whose primary purpose is to hold, support, maintain and fixate one or more other distinct elements or device within the eye, where the device serves a biologically important function;
“Haptic Passenger” refers to a functionally important device supported by the haptic, examples of Haptic Passengers and their associated functions include an optical lens system, a reservoir, depot or container for a therapeutic substance or drug, a diagnostic instrument or sensor;
“IOL haptic” or refers to a structural element of an IOL designed to hold an IOL in place within the eye, such as a haptic whose haptic passenger is a lens;
“IOL optic” refers to the optically active component of the IOL having light transmitting refractive power, such as the haptic passenger for an IOL haptic;
“Capsular bag” or “bag” refers to the partially elastic biological membrane which normally contains the lentil shaped crystalline lens of the eye between a front surface (anterior capsule) and a back surface (posterior capsule) which join at the equator of the capsular bag from which equator the lens is suspended from and connected to the processes of the ciliary body by zonules (or suspensory ligaments of the lens), the capsular bag is opened during cataract surgery to remove the cataractous lens by making a roughly circular opening in its anterior capsule, the capsular bag has traditionally been the desired location in which to place an IOL after cataract extraction, the IOL is normally placed through the anterior capsular opening or “rhexis” so that its spring like supporting haptics rest in or close to the equator of the bag, suspending the optic of the IOL within and perpendicular to the visual axis;
“Capsulorhexis” or “rhexis” refers to the surgical opening made in the capsular bag and is a vital step in modern cataract surgery, it is necessary to access the cataract for removal and to insert an IOL if it is to be placed in the capsular bag, and the terms “rhexis” and “incision” are used interchangeably herein;
“Posterior capsular fibrosis” or “Posterior capsular opacification” (PCO) refers to the migration and proliferation of fibroblast inside and around the remnants of the capsular bag following cataract surgery, in addition to reducing vision, the scar tissue formed by these fibroblasts causes scarring and contracture of the capsular bag resulting in loss of its elastic properties, posterior capsular fibrosis occurs to at least some extent in the majority of patients following cataract despite various precautions commonly taken to reduce it, contracture of the capsular bag can cause tilt or displacement of an IOL in contact with the bag and will limit post-operative capsular bag movement in response to CBA, the severity of posterior capsular fibrosis is unpredictable but often warrants YAG laser capsulotomy after surgery to break open the capsule when it interferes with vision, the behavior of the capsular remnants following YAG laser capsulotomy is even more unpredictable, this means that any AIOL that relies on capsular bag contraction for functioning is unlikely to be successful because CBA cannot be reliably translated into IOLA by the post-surgical capsular bag;
“Accommodating IOL” or “AIOL” of Intraocular Optic Assembly refers to a prosthetic lens or IOL that seeks to restore the function of lenticular accommodation (other than by pseudo-accommodation or monovision) in a patient whose crystalline lens has been removed;
“Simple lens” refers to the concave and convex cross sections depicted in optical drawings and ray diagrams shown commonly in physics textbooks, wherein the convex or concave surfaces enclose a medium whose refractive index is different to that of the media in front and behind the lens, although its front and rear surfaces are separated such a lens has a point (which can actually lie outside the body of the lens) known as the optical center of the lens whose location and optical properties can be described in an idealized fashion by “Thin Lens Theory”, and in a more complex, and potentially more accurate fashion by “Thick Lens Theory”, the power of such a lens is normally fixed and does not change because the lens is solid and static, the power of a particular simple lens can be made different to that of another by altering one or both of the front and rear curvatures or the refractive index of the medium behind and/or in front of the lens;
“Compound lens” refers to a lens composed of two or more simple lenses whose overall optical parameters can be varied by varying the power of each component lens, varying the separation between the optical centers of the component lenses, and varying other spatial relationship (such as tilt and alignment) between the optical centers or surfaces of the component lenses;
“Flexible lens” refers to a lens composed of an optical medium which is fluid or gel like in mechanical property, and of essentially constant volume, and whose volume is contained and bounded across at least part of its surface by an elastic or flexible membrane, the power of a flexible lens can be varied by shape change of the fluid or gel like medium when such shape changes result in variations in curvature of the flexible membrane when the membrane lies across the visual axis, variation in separation of the front and back surfaces, and variation in location of optical center of lens;
“Biological lens” refers to a lens with front and back surfaces whose body is composed of regions of varying refractive index without clear demarcation or interface between the zones, the regions may be distributed so that the gradient in refractive index varies perpendicular to its optic axis (refractive index changing from center to periphery in a concentric radial fashion) and/or varies in the line of the optic axis so that the refractive index is maximum at the front surface, back surface or center of the lens, variations of the power of a biological lens can be achieved by a spatial redistribution of the regions of high and low refractive indices and may be achieved by overall change in the shape of the lens when it is contained within a flexible membrane or redistribution of the optical centers of the regions of different refractive index without overall shape change of the external boundaries of the lens capsule, resulting in a shifting of the optical center of the lens;
“Neo-biological lens” refers to a lens composed of material whose refractive index can be varied be electronic or photo-chemical means either across the entire material of the lens, or selectively in certain regions; and
“Higher Order Aberrations” or “HOA” relates to imperfections of focusing of a nature more complex than lower order optical aberrations such as spherical error and astigmatism, clinically important examples of HOA include spherical aberration, coma and trefoil, correction of HOA can improve visual quality and satisfaction following ocular surgery.

The exact nature and relative importance of various physiological mechanisms active in the human eye during the act of accommodation is controversial. The theory of Helmholtz appears to be the most favored. It is agreed that contractions of the ciliary body/muscle occur in response to neural signals from the brain when accommodation is voluntarily or reflexly initiated. It is also agreed that in the youthful eye, this contraction causes several mechanical changes that result in the optical diopteric power of the lens system becoming more positive and so shifting the focal point of the lens closer to the person. The optical power change is thought to result from an anterior shift of the overall optical center of the lens closer to the cornea and an increase in curvature of the anterior and/or posterior refracting surfaces of the lens (necessitated by the requirement to maintain constant volume within the enclosing capsular bag) when the lentil shaped lens decreases in circumference at its attachment points (zonular fibers) in the plane roughly perpendicular to the visual axis.

In practice, other subtle changes may also contribute to a lesser extent such as constriction of the pupil to induce a pin-hole effect to increase depth of field-pseudo accommodation, shift of the constricted pupillary center away from the relaxed pupillary center to preferentially select a new optical line of site within the eye of different refractive power, and change in lens shape may cause shifting of relative position within the lens, of areas of differing pliability, elasticity and refractive index to cause a change in overall power.

For AIOL design a clear understanding of the anatomical changes occurring in the eye during CBA is desirable. In some species, CBA results in muscular activity that alters the curvature of the cornea or the length of the eyeball amongst other changes, but in humans, alterations of the shape and location of the crystalline lens appear to be the main mediators of accommodation.

When CBA is initiated in humans, at least three muscular sub systems within the ciliary body are activated. First, there is an annular or circular component—a sphincter muscle in the shape of a toroid in a plane approximately perpendicular to the visual axis, located internally to the scleral coat of the eye within the partially elastic parenchyma or connective tissue of the CB. This annular component contracts on accommodation so that the toroid becomes smaller in diameter and thicker in its cross section while the plane of the toroid moves closer to the front of the eye in the line of the visual axis. This contraction releases tension on the lens zonules and capsular bag, thereby causing forward movement of the optical center of the lens and a reduction in the equatorial diameter of the lens capsule.

Second, meridional or longitudinal components that run in approximately parallel to each other slight curve under the sclera connection their relatively stationary attachment on the sclera at one end to the pars plana of the ciliary body at the other end. The effect of contraction of these fibers is to pull the area of attachment of lens zonules anteriorly along the interior surface of the eyeball as it approaches the cornea. The anatomy of the anterior eyeball is such so that this movement results in release in tension of the lens zonules, especially those connecting to the front surface of the lens capsule so that the lens returns to a more rounded shape and its optical center moves forward. The annular fibers of the ciliary muscle lie in a ring separated from the sclera and eyeball by the longitudinal fibers so that the contraction of the longitudinal fibers mechanically facilitates the contraction of the annular components by occupying and increasing the space between the outer aspect of the ring muscles and the sclera.

Third, oblique fibers that run a semi-spiral course under the sclera of the eyeball. They likely act as slings to reduce forces that might inwardly detach the pars plana of the ciliary body and prevent wrinkling of the pars plana of the ciliary body during CBA.

Although the ciliary muscle is usually depicted in cross section, it is actually a complex 3-D structure that is fixed at its outside margin to the sclera of the eyeball and whose inside margin suspends the zonules which connect to the capsular bag. Different species have at least three types of muscle fibers within the ciliary muscle. The exact contribution of the various mechanisms linked to accommodation are not fully known but for the purpose of at least some embodiments of the present disclosure the important points are that when contracted during accommodation the ciliary muscle concentrates into a toroid which decreases in inside diameter, increases in cross sectional area, and moves forward in the plane perpendicular to visual axis with regards to the location of its center of volume.

Contraction of the ciliary muscle leads to changes in the three-dimensional shape of the lens capsule as well as displacement of the optical center of the lens in relation to the overall optical center of the eye itself. This displacement alters the overall focal point of the eye allowing variability of focus from distance to near objects.

When accommodation is relaxed in the human eye, outward radial pull via tension in the suspensory ligaments (zonules) of the lens leads to an increase in the circular diameter of the space contained within the lens capsule in the plane approximately perpendicular to the visual axis and path of light from distant objects to the central retina of the eye. The act of accommodation causes the ciliary muscle of the eye to contract which releases tension in the suspensory lens ligaments resulting in reduced diameter of the lens in the visual plane and changes in the anterior and posterior surface curvatures of the lens as well as shifting of the optical center of the lens which result in increased convex diopteric power of the lens and consequently of the whole optical system of the eye allowing near objects to be focused on the retina.

The crystalline lens of the eye is normally flexible and is suspended within an elastic capsule. This capsule has to be penetrated to remove the cataractous lens.

The shape of the lens capsule and enclosed lens in its natural state depends on the interaction between the elastic nature of the capsule and also (a) the tension in the supporting zonules whose force and direction is varied by contraction of the ciliary muscle, (b) resistance and pressure from the vitreous humor against the posterior capsule surface, (c) forces on the anterior surface of the lens capsule from aqueous humor and iris, (d) gravity, and (e) resistance to deformity of the contents of the lens capsule, normally the crystalline lens.

One or more embodiments of the present disclosure utilize biometric changes occurring during CBA. The primary biometric changes utilized are reductions in the sulcus-to-sulcus diameter (SSD), the anterior chamber depth (ACD), the iris-ciliary process angle (ICPA), and the iris-zonula distance (IZD, or posterior chamber depth). Indirect or secondary biometric changes occurring during CBA that can be utilized in one or more embodiments of the present disclosure include reductions in the ciliary process-capsular bag distance (CP-CBD) decreases and the ciliary ring diameter (CRD).

Although there is considerable variability in the exact measured mean values for the various anatomical distance and angles compared in the relaxed and near accommodated state, this is not surprising given the normal anatomical variations between studied individuals as well as the variety of instruments and techniques used in different studies. Additionally, the resolution of the current technology is still sub optimal, as are agreements in precise location of landmarks. Because of the above-mentioned factors, comparison of the various studies shows a wide variability of the mean measured values in both the relaxed and near accommodated state, as well as large standard deviations in the mean difference values. This results in low confidence in the statistical significance of the mean differences in many of the studies. However, at least some embodiments of the present disclosure assume that there are some consistent and predictable variations in measured anatomical parameters during near accommodation including (a) a decrease in the SSD (sulcus-to-sulcus diameter) from approximately 11 mm to approximately 10.5 mm, (b) a decrease in the ICPA (Iris-ciliary process angle) from approximately 40 degrees to approximately 22 degrees, (c) a decrease in the ACA (anterior chamber angle) from approximately 32 degrees to approximately 28 degrees, (d) a decrease in the distance from the ciliary sulcus to the apex of the cornea caused by movement of the plane of the ciliary sulcus anteriorly along the visual axis, and (e) an increase in the diameter of the circular portion of the ciliary muscle. One or more embodiments of the present disclosure can use the above anatomical changes to mechanically link CBA to IOLA in a manner superior to the prior art.

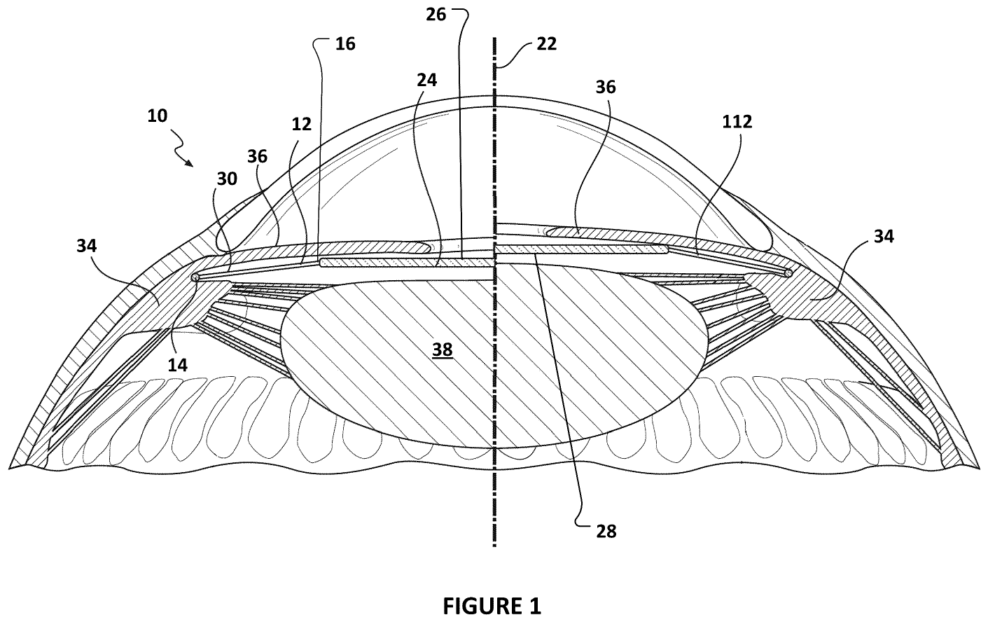

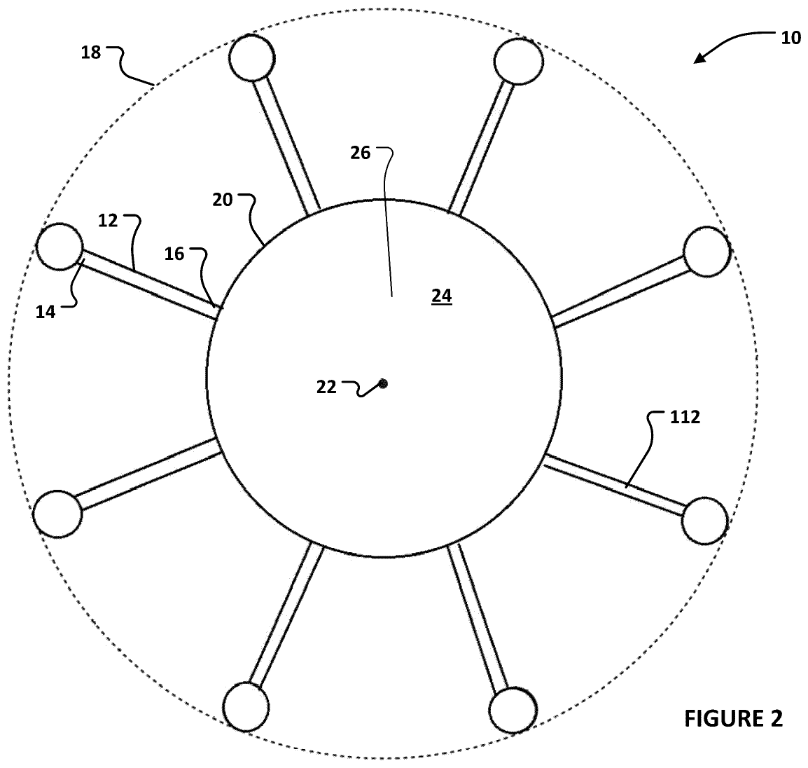

FIG. 1 is a cross-sectional side view of a first exemplary accommodating intraocular optic assembly 10 in a first exemplary operating environment in which features disclosed herein can be utilized. FIG. 2 is a front view of the first exemplary accommodating intraocular optic assembly 10. The exemplary accommodating intraocular optic assembly 10 includes an optic in the form of a positive power lens 24 and a plurality of stanchions, such as stanchions 12, 112, supporting the lens 24. Each of the plurality of stanchions can extend between a base end and a distal end. The stanchion 12 extends a length from a base end 14 and a distal end 16. The respective base ends of the plurality of stanchions can be disposed in spaced relation to one another about a first arcuate periphery 18 extending in a first plane. The respective distal ends of the plurality of stanchions can be disposed about a second arcuate periphery 20 extending in a second plane spaced from the first plane along a central optic axis 22. The first arcuate periphery 18 and the second arcuate periphery 20 can both be centered on the optic axis 22. The exemplary first arcuate periphery 18 is positioned in the ciliary sulcus 30 and each of the respective base ends can be bulbous and/or at least partially spherical. The plurality of stanchions can extend away from the base ends and the first arcuate periphery 18 toward the distal ends and the second arcuate periphery 20.

The lens 24 can have an anterior side 26 and a posterior side 28 and a center disposed between the anterior side 26 and the posterior side 28. The positive-power lens 24 can be connected with each of the plurality of distal ends whereby the center of the positive power lens 24 is moved along the central optic axis 22 in the anterior direction in response to contraction of the first arcuate periphery 18 by contraction of the ciliary muscle 34. Upon relaxation of the ciliary muscle 34, the center of the lens 24 moves along the central optic axis 22 in the posterior direction.

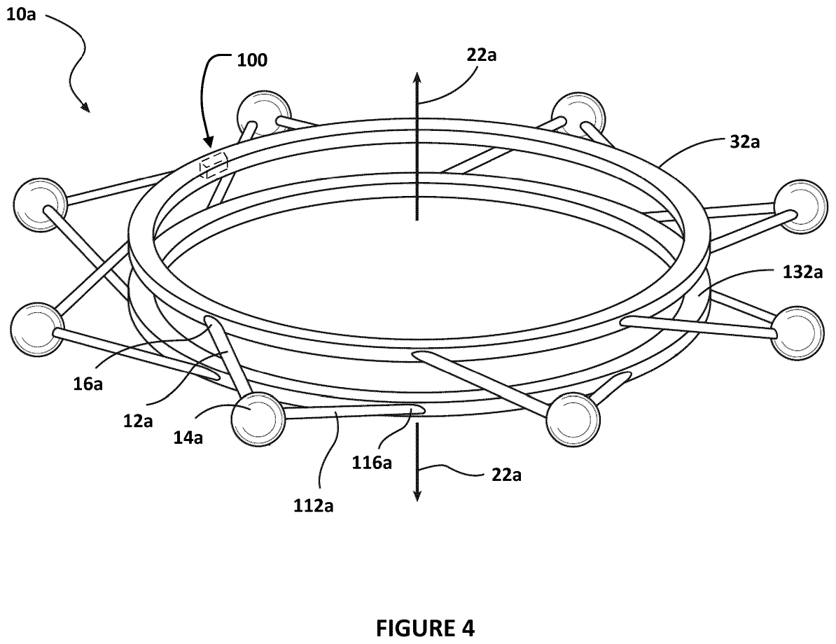

In one or more embodiments of the present disclosure, the lens 24 can be directly connected to the stanchions or can be indirectly connected to the stanchions. In an embodiment applying indirect connection, the lens 24 can be mounted in a ring member and the distal ends of the stanchions can be connected to the ring member. An exemplary ring member is referenced at 32a in FIG. 4 and will be described in greater detail below. The exemplary distal ends 16 of the assembly 10 are directly, operably engaged with the lens 24. For example, the distal ends 16 of the assembly 10 can be connected to the lens 24 with adhesive. A lens is one form of optic, a ring member without a lens but placed in the eye is another form of optic, and the combination of a ring member and a lens mounted on the ring member is another form of optic.

FIG. 1 is a split cross-sectional view showing the accommodating intraocular optic assembly 10 according to the first exemplary embodiment of the present disclosure position in an eye. The lens 24 of the accommodating intraocular optic assembly 10 is positioned between an iris 36 and a capsular bag 38. The left side of the view of FIG. 1 shows the ciliary muscle 34 in the relaxed condition and the right side of the view shows the ciliary muscle 34 in the contracted condition. In an exemplary operation of the first exemplary embodiment, when the ciliary muscle 34 is relaxed, the lens 24 is disposed at a first position within the eye and the stanchion 12 is disposed at a first angle relative to the lens 24. When the ciliary muscle 34 contracts, the lens 24 is moved to a second position in the eye, the second position being anterior to the first position.

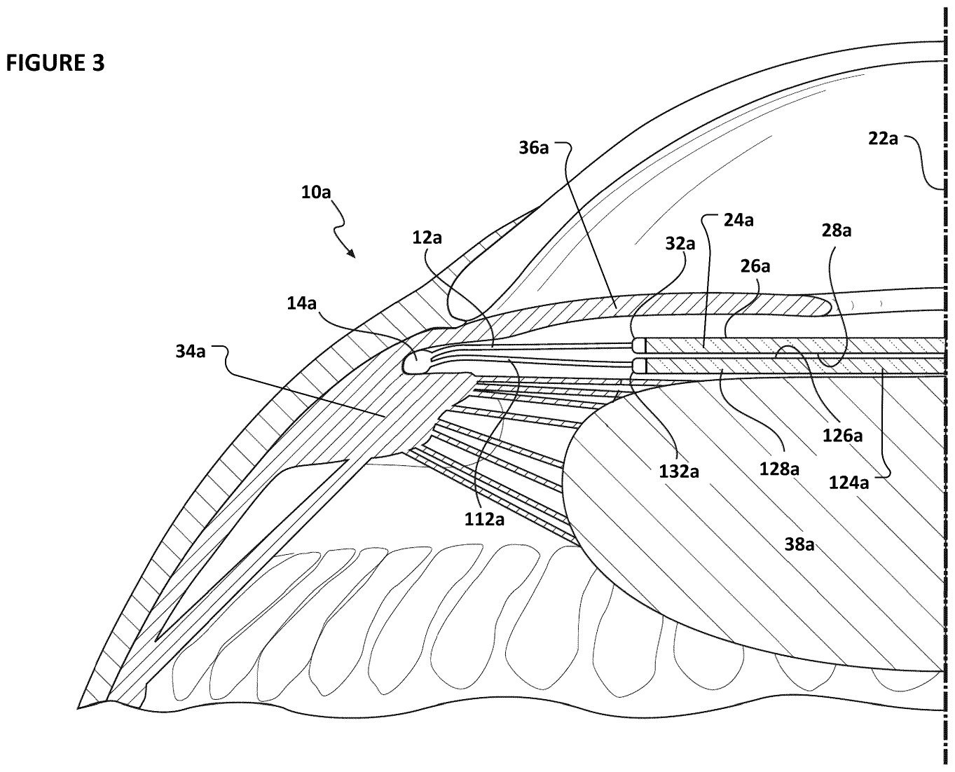

FIG. 3 is a cross-sectional side view of a portion of second exemplary accommodating intraocular optic assembly 10a in a second exemplary operating environment in which features disclosed herein can be utilized. FIG. 4 is a perspective view of the second exemplary accommodating intraocular optic assembly 10a. The exemplary accommodating intraocular optic assembly 10a includes a pair of lenses 24a, 124a and the lenses 24a, 124a are respectively mounted in ring members 32a and 132a. The exemplary distal ends 16a of the assembly 10a are indirectly, operably engaged with the lens 24a through the ring member 32a. For example, the distal ends of the stanchions 12a, 112a of the assembly 10a can be respectively connected to the ring members 32a, 132a with adhesive. The exemplary distal ends of the stanchions 112a of the assembly 10a are indirectly, operably engaged with the lens 124a through the ring member 132a. The exemplary lens 24a includes an anterior side 26a and a posterior side 28a. The exemplary lens 124a includes an anterior side 126a and a posterior side 128a.

The exemplary accommodating intraocular optic assembly 10a also includes a plurality of stanchions, such as stanchions 12a, 112a. Each of the plurality of stanchions can extend between a base end and a distal end. The exemplary stanchion 12a extends a length from a base end 14a and a distal end 16a. The exemplary stanchion 112a extends from the base end 14a and a distal end 116a. The stanchions 12a and 112a thus share the base end 14a. The distal end 16a is connected to the ring 32a and the distal end 116a is connected to the ring 132a.

The lenses 24a, 124a are both positioned between an iris 36a and a capsular bag 38a. The lens 24a can be operably engaged with each of the plurality of distal ends 16a whereby a center of the lens 24 is moved along a central optic axis 22a in the anterior direction in response to contraction of the base ends 14a by contraction of the ciliary muscle 34. The lens 124a can be operably engaged with each of the plurality of distal ends 116a whereby a center of the lens 124a is moved along the central optic axis 22a in the posterior direction in response to contraction of the base ends 14a by contraction of the ciliary muscle 34a. Upon relaxation of the ciliary muscle 34a, the center of the lens 24a moves along the central optic axis 22a in the posterior direction and the center of the lens 124a moves along the central optic axis 22a in the anterior direction.

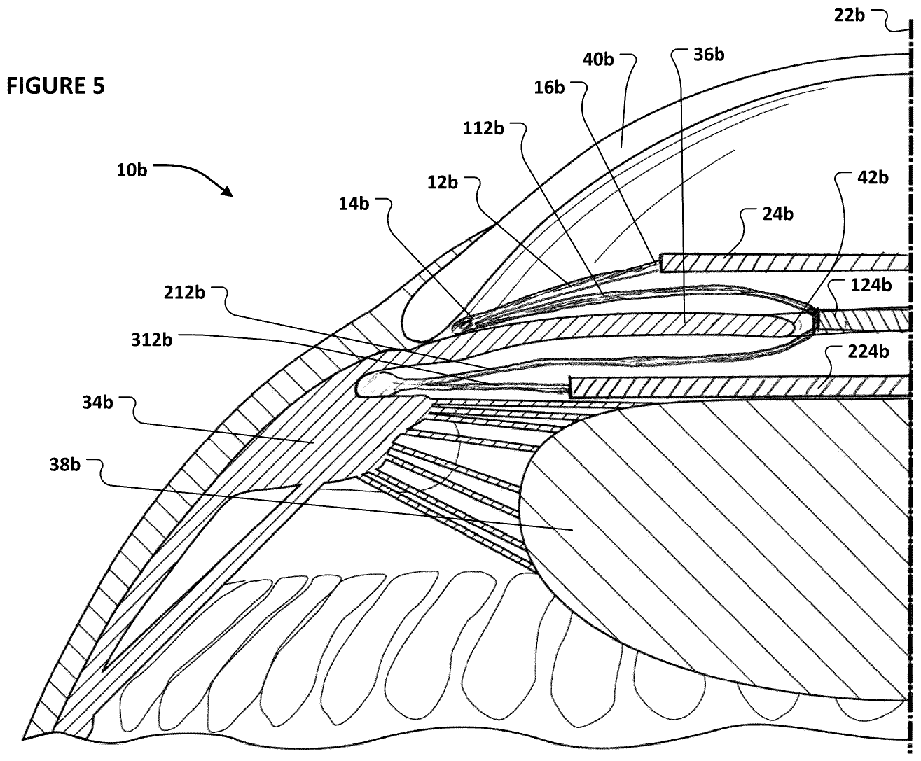

FIG. 5 is a cross-sectional side view of a portion of third exemplary accommodating intraocular optic assembly 10b in a third exemplary operating environment in which features disclosed herein can be utilized. The exemplary accommodating intraocular optic assembly 10b includes three lenses 24b, 124b, 224b. The exemplary accommodating intraocular optic assembly 10b also includes a plurality of stanchions, such as stanchions 12b, 112b, 212b, 312b. Each of the plurality of stanchions can extend between a base end and a distal end. The exemplary stanchion 12b extends a length from a base end 14b and a distal end 16b.

The exemplary lens 24b is positioned between a cornea 40b and an iris 36b. The exemplary lens 124b is positioned in the pupil 42b. The exemplary lens 224b is positioned between the iris 36b and a capsular bag 38b.

The exemplary lens 24b can be operably engaged with each of the plurality of distal ends of the stanchions 12b whereby the center of the lens 24b moves along a central optic axis 22b in response to contraction and relaxation of a ciliary muscle 34b. For example, the lens 24b can move in the anterior direction in response to contraction of the base ends of the stanchions 12b by contraction of the ciliary muscle 34b. Contraction of the base ends of the stanchions 12b refers to movement of the base ends of the stanchions 12b toward the optic axis 22b. Upon relaxation of the ciliary muscle 34b, the center of the lens 24b moves along the central optic axis 22b in the posterior direction.

The exemplary lens 224b can be operably engaged with each of the plurality of distal ends of the stanchions 312b whereby a center of the lens 224b is moved along the central optic axis 22b in the posterior direction in response to contraction of the base ends of the stanchions 312b by contraction of the ciliary muscle 34b. Upon relaxation of the ciliary muscle 34b, the center of the lens 224b moves along the central optic axis 22b in the anterior direction.

The exemplary lens 124b can be operably engaged with each of the plurality of distal ends of the stanchions 112b and 212b. The exemplary lens 124b can generally remain in the pupil 42b during contraction and relaxation of the ciliary muscle 34b. The contraction of the base ends of the stanchions 12b toward the optic axis 22b and the resulting elastic deformation of the stanchions 12b that moves the lens 24b can be offset by the contraction of the base ends of the stanchions 312b toward the optic axis 22b and the resulting elastic deformation of the stanchions 312b that moves the lens 224b. This arrangement beneficially allows for a relatively small amount of contraction to yield relatively larger amounts of movement of the lenses 24b and 224b.

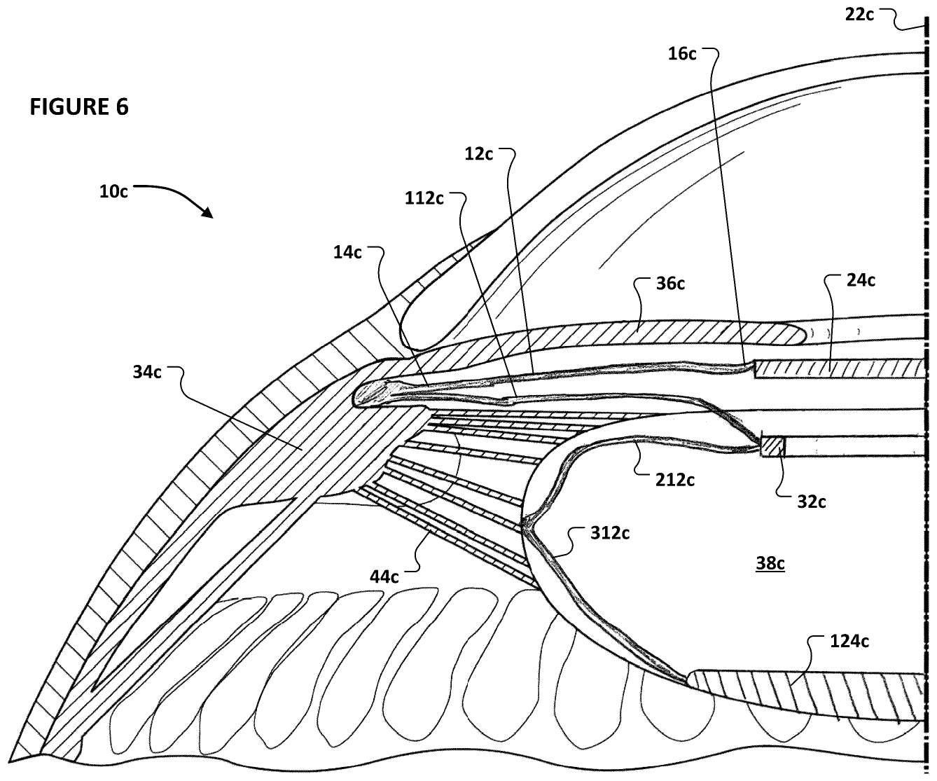

FIG. 6 is a cross-sectional side view of a portion of the fourth exemplary accommodating intraocular optic assembly 10c in a fourth exemplary operating environment in which features disclosed herein can be utilized. The exemplary accommodating intraocular optic assembly 10c includes two lenses 24c, 124c and a ring member 32c. The exemplary ring member 32c does not support another lens/optic. The exemplary accommodating intraocular optic assembly 10c also includes a plurality of stanchions, such as stanchions 12c, 112c, 212c, 312c. Each of the plurality of stanchions can extend between a base end and a distal end. The exemplary stanchion 12c extends a length from a base end 14c and a distal end 16c.

The exemplary lens 24c is positioned between an iris 36c and a capsular bag 38c. The exemplary lens 124c and ring 32c are positioned in the capsular bag 38c.

The exemplary lens 24c can be operably engaged with each of the plurality of distal ends of the stanchions 12c whereby a center of the lens 24c moves along a central optic axis 22c in response to contraction and relaxation of a ciliary muscle 34c. For example, the lens 24c can move in the anterior direction in response to contraction of the base ends of the stanchions 12c by contraction of the ciliary muscle 34c. Contraction of the base ends of the stanchions 12c refers to movement of the base ends of the stanchions 12c toward the optic axis 22c. Upon relaxation of the ciliary muscle 34c, the center of the lens 24c moves along the central optic axis 22c in the posterior direction.

The exemplary lens 224c can be operably engaged with each of the plurality of distal ends of the stanchions 312c whereby a center of the lens 224c is moved along the central optic axis 22c in the posterior direction in response to contraction of the base ends of the stanchions 312c by contraction of the ciliary muscle 34c. Contraction of the ciliary muscle 34c will reduce stretching tension placed on the capsular bar 38c by zonules, such as zonule 44c. The capsular bag 38c will then contract and urge the base ends of the stanchions 212c and 312c toward the axis 22c. This will result in movement of the lens 124c in the posterior direction. Upon relaxation of the ciliary muscle 34c, the center of the lens 224c moves along the central optic axis 22c in the anterior direction.

The exemplary ring member 32c can be operably engaged with each of the plurality of distal ends of the stanchions 112c and 212c. The exemplary ring member 32c can generally remain in the same position during contraction and relaxation of the ciliary muscle 34c. The contraction of the base ends of the stanchions 12c toward the optic axis 22c and the resulting elastic deformation of the stanchions 12c that moves the lens 24c can be offset by the contraction of the base ends of the stanchions 312c toward the optic axis 22c and the resulting elastic deformation of the stanchions 312c that moves the lens 224c. This arrangement beneficially allows for a relatively small amount of contraction to yield relatively larger amounts of movement of the lenses 24c and 224c.

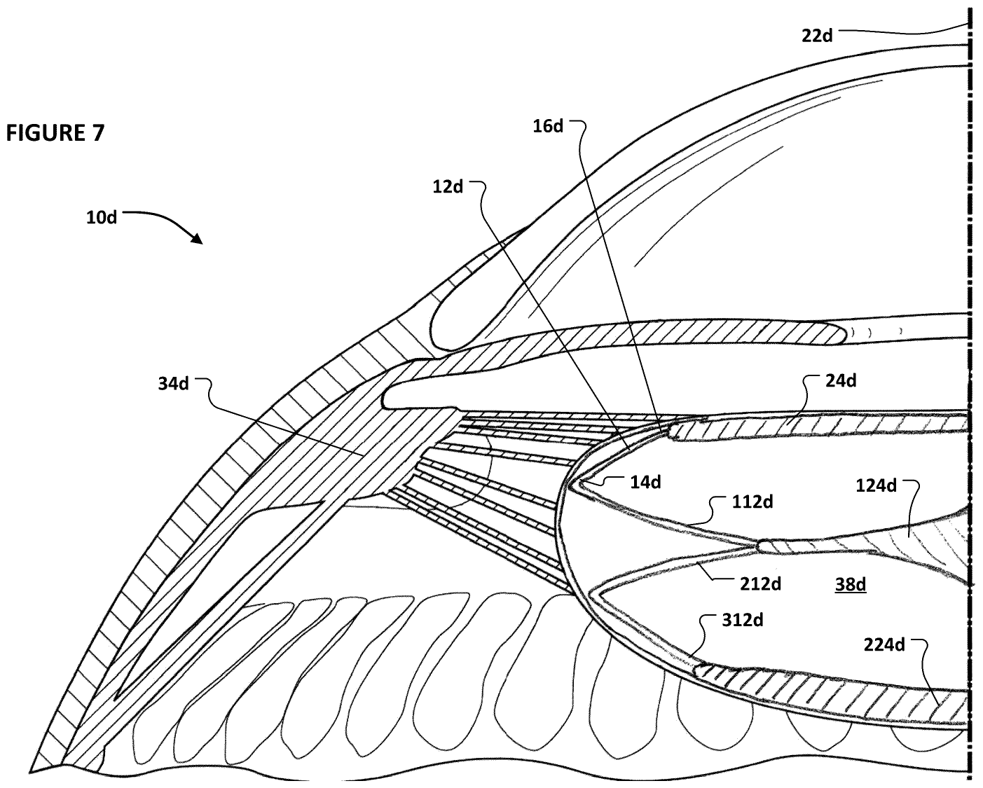

FIG. 7 is a cross-sectional side view of a portion of the fifth exemplary accommodating intraocular optic assembly 10d in a fifth exemplary operating environment in which features disclosed herein can be utilized. The exemplary accommodating intraocular optic assembly 10d includes three lenses 24d, 124d, 224d. The exemplary accommodating intraocular optic assembly 10d also includes a plurality of stanchions, such as stanchions 12d, 112d, 212d, 312d. Each of the plurality of stanchions can extend between a base end and a distal end. The exemplary stanchion 12d extends a length from a base end 14d and a distal end 16d.

The exemplary lenses 24d, 124d, 224d are in a capsular bag 38d. The exemplary lens 24d is operably engaged with each of the plurality of distal ends of the stanchions 12d whereby the center of the lens 24d moves along a central optic axis 22d in response to contraction and relaxation of a ciliary muscle 34d. For example, the lens 24d can move in the anterior direction in response to contraction of the base ends of the stanchions 12d by contraction of the ciliary muscle 34d. Contraction of the base ends of the stanchions 12d refers to movement of the base ends of the stanchions 12d toward the optic axis 22d. Contraction of the ciliary muscle 34d will reduce stretching tension placed on the capsular bar 38d by zonules, such as zonule 44d. The capsular bag 38d will then contract and urge the base ends of the stanchions 12d, 112d, 212d, 312d toward the axis 22d. Upon relaxation of the ciliary muscle 34d, the center of the lens 24d moves along the central optic axis 22d in the posterior direction. Upon, or in response to, contraction of the ciliary muscle 34d the center of the lens 224d moves along the central optic axis 22d in the posterior direction and upon relaxation the center of the lens 224d moves along the central optic axis 22d in the anterior direction. The exemplary lens 124d can generally remain in the same position during contraction and relaxation of the ciliary muscle 34d.

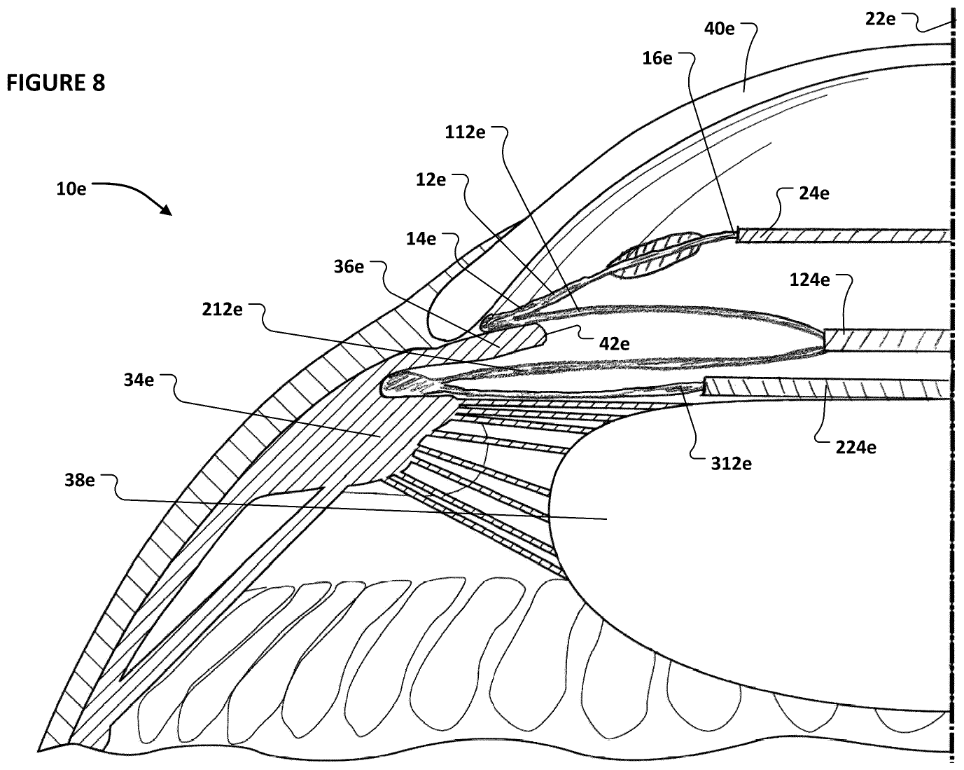

FIG. 8 is a cross-sectional side view of a portion of sixth exemplary accommodating intraocular optic assembly 10e in a sixth exemplary operating environment in which features disclosed herein can be utilized. The exemplary accommodating intraocular optic assembly 10e includes three lenses 24e, 124e, 224e. The exemplary accommodating intraocular optic assembly 10e also includes a plurality of stanchions, such as stanchions 12e, 112e, 212e, 312e. Each of the plurality of stanchions can extend between a base end and a distal end. The exemplary stanchion 12e extends a length from a base end 14e and a distal end 16e.

The exemplary lens 24e is positioned generally between a cornea 40e and an iris 36e along a central optic axis 22e. The exemplary lens 124e is positioned generally in the pupil 42e. The exemplary lens 224e is positioned generally between the iris 36e and a capsular bag 38e along the central optic axis 22e.

The exemplary lens 24e can be operably engaged with each of the plurality of distal ends of the stanchions 12e whereby the center of the lens 24e moves along the central optic axis 22e in response to contraction and relaxation of a ciliary muscle 34e. For example, the lens 24e can move in the anterior direction (along the axis 22e) in response to contraction of the base ends of the stanchions 12e by contraction of the ciliary muscle 34e. Contraction of the base ends of the stanchions 12e refers to movement of the base ends of the stanchions 12e toward the central optic axis 22e. Upon relaxation of the ciliary muscle 34e, the center of the lens 24e moves along the central optic axis 22e in the posterior direction.

The exemplary lens 224e can be operably engaged with each of the plurality of distal ends of the stanchions 312e whereby a center of the lens 224e is moved along the central optic axis 22e in the posterior direction in response to contraction of the base ends of the stanchions 312e by contraction of the ciliary muscle 34e. Upon relaxation of the ciliary muscle 34e, the center of the lens 224e moves along the central optic axis 22e in the anterior direction.

The exemplary lens 124e can be operably engaged with each of the plurality of distal ends of the stanchions 112e and 212e. The exemplary lens 124e can generally remain in the pupil 42e during contraction and relaxation of the ciliary muscle 34e. The contraction of the base ends of the stanchions 112e toward the optic axis 22e and the resulting elastic deformation of the stanchions 112e that moves the lens 24e can be offset by the contraction of the base ends of the stanchions 212e toward the optic axis 22e and the resulting elastic deformation of the stanchions 312e that moves the lens 224e. This arrangement beneficially allows for a relatively small amount of contraction to yield relatively larger amounts of movement of the lenses 24e and 224e.

In the exemplary accommodating intraocular optic assemblies disclosed above the stanchions are represented in the Figures as generally homogenous and thus consistently/similarly flexible along the respective stanchion's entire length. Along relatively straight sections of each stanchion, the capacity to elastically deform can be the generally same. For stanchions that include a bend or kink along its length, such as stanchion 12a in FIG. 3 near the base end 14a, the bend or kink will alter bending characteristics but otherwise the respective stanchion's bending characteristics are generally the same along the remainder of its entire length.

The various accommodating intraocular optic assemblies disclosed above can be inserted into an eye of a patient by wrapping or winding the stanchions around a lens or ring and folding the “wound” assembly in half (such as “taco” shape). For example, in FIG. 2, the base end 14 and the other base ends can be elastically deformed by being bent around the lens 24, clockwise or counterclockwise, and then the assembly 10 can be folded. In this wound and folded configuration, the assembly can be placed in a holding tool, an exit port of the tool can be positioned in the eye, and the wound and folded assembly can be directed out of the holding tool and into the eye. Upon insertion, the respective assembly can unfold and unwind to change into one of the respective configurations shown in the FIGS. 1-8.

The present disclosure provides improvements in accommodating intraocular optic assemblies in the form of a stanchion configured to provide selective, post implant stiffening. The improvements allow a stanchion to still be easily wound and foldable but also be stiff and more resistant to bending subsequent to the unwinding after insertion. Stiffening can be selective in that an improvement can be defined at a particular position along the length of the stanchion.

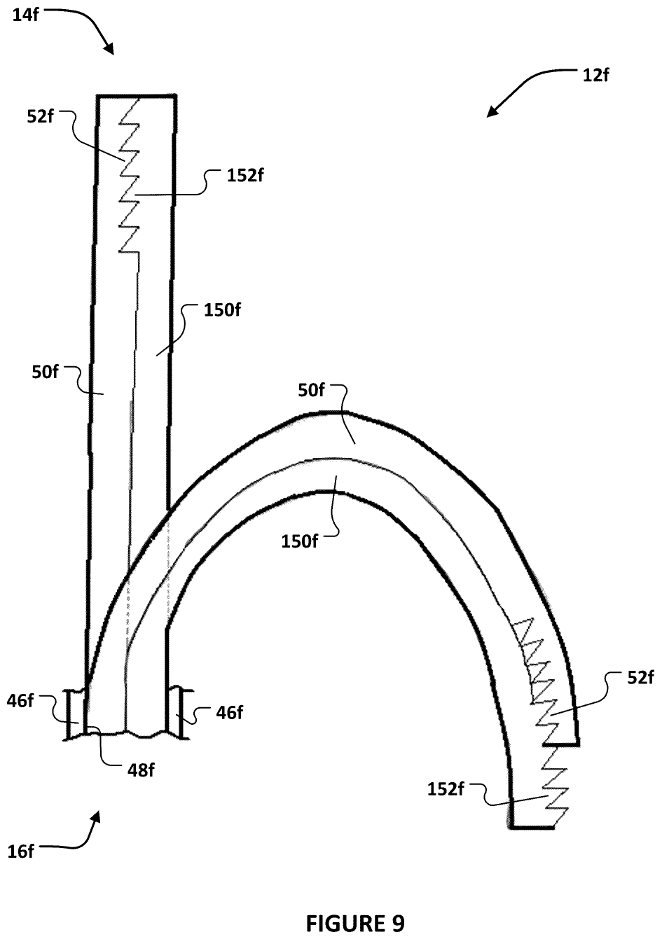

FIG. 9 is a side view of a first embodiment of an improvement in accommodating intraocular optic assemblies in the form of a stanchion 12f configured to provide selective, post implant stiffening. Any of the accommodating intraocular optic assemblies disclosed herein as well as any accommodating intraocular optic assemblies not disclosed herein can incorporate the stanchion 12f. The stanchion 12f can extend a length between a base end 14f and a distal end 16f. The distal end 16f can be operably engaged with an optic, such as lens, directly or indirectly, as disclosed and shown in the various accommodating intraocular optic assemblies shown in FIGS. 1-8. The exemplary stanchion 12f includes an outer sleeve 46f defining a through-aperture 48f. The exemplary stanchion 12f also includes at least one inner member positioned within the through-aperture 48f It is noted that in FIG. 9, the sleeve 46f is shown partially cut-off at the point of bending to enhance the clarity of the remaining structures in the Figure but can extend the full length of the stanchion 12f Also, in FIG. 9, the distal end 16f is cut-off to permit the showing of the stanchion 12f to be as large as possible.

The exemplary at least one inner member of the stanchion 12f includes a first elongate member 50f defining a first set of rachet teeth 52f and a second elongate member 150f defining a second set of rachet teeth 52f The exemplary first elongate member 50f and the exemplary second elongate member 150f are configured to slide across one another when the stanchion 12f bends in a first direction. Based on the orientation of FIG. 9, the exemplary first direction that is shown is a clockwise direction. Figure includes a view of the stanchion 12f bent overlapped with a view of the stanchion 12f unbent. It is also noted that the while the sleeve 46f is not shown over the bent portion of the stanchion 12f, the exemplary sleeve 46f would cover at least part of the bent portion of the stanchion 12f.

The first set of rachet teeth 52f and the second set of rachet teeth 152f are configured to slide across one another when the at least one stanchion 12f bends in the first direction. This is shown in FIG. 9 by the respective portions of the base end 14f respectively defined by the elongate members 50f, 150f being aligned when the stanchion 12f is unbent (top left of the view) and unaligned when the stanchion 12f is bent (bottom right of the view).

The first set of rachet teeth 52f and the second set of rachet teeth 152f are further configured to lock together when the stanchion 12f bends in a second direction that is opposite to the first direction. As shown in the bottom right of the view, the sets of rachet teeth 52f, 152f have locked and the elongate members 50f, 150f, are prevent from sliding back across one another to return to the original configuration (shown in FIG. 9 as straight up and down). In FIG. 9, sets of rachet teeth 52f, 152f are engaged with one another in the original configuration, but it is noted that in other embodiments opposing sets of rachet teeth may not be engaged with one another in the original configuration but may come into engagement only after the stanchion has been at least partially bent.

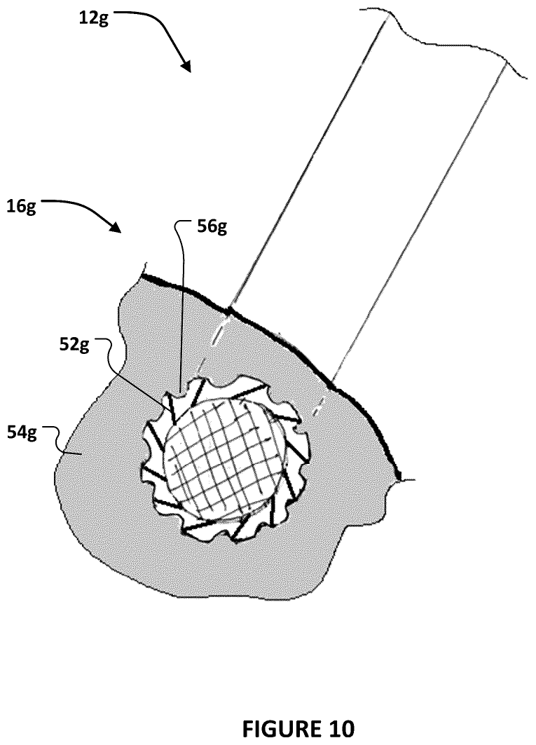

FIG. 10 is a detail view of a second embodiment of an improvement in accommodating intraocular optic assemblies in the form of a portion of a stanchion 12g configured to provide selective, post implant stiffening. Any of the accommodating intraocular optic assemblies disclosed herein as well as any accommodating intraocular optic assemblies not disclosed herein can incorporate the stanchion 12g. The exemplary stanchion 12g includes rachet teeth 52g that are flap-like at either the base end or the distal end. A structure referenced at 54g can be an optic at the distal end 16g of the stanchion 12g, such as a lens or a ring, and can define protuberances 56g that act as mating rachet teeth with rachet teeth 52g. The exemplary stanchion 12g can thus rotate relative to the structure 54g in only one direction. It is also noted that the rachet arrangement can be defined at the base end of the stanchion 12g as well or in alternative to being defined at the distal end.

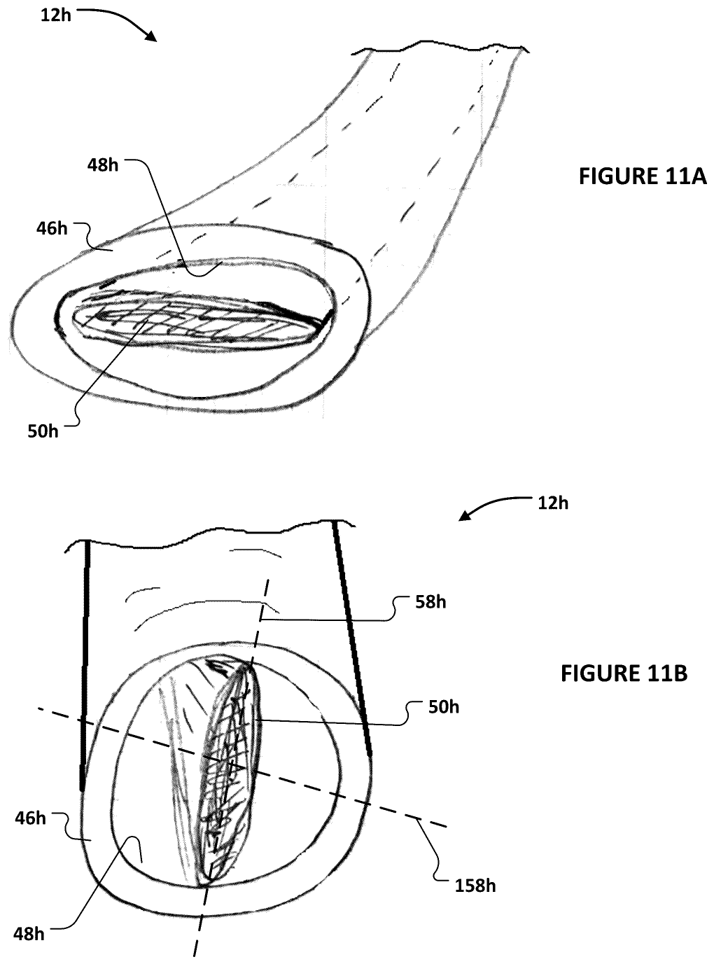

FIGS. 11A and 11B are perspective views of a third embodiment of an improvement in accommodating intraocular optic assemblies in the form of a stanchion 12h configured to provide selective, post implant stiffening. Any of the accommodating intraocular optic assemblies disclosed herein as well as any accommodating intraocular optic assemblies not disclosed herein can incorporate the stanchion 12h. The stanchion 12h can extend a length between a base end and a distal end. It is noted that FIGS. 11A and 11B show a portion of the stanchion 12h between the base and distal ends. The distal end of the stanchion 12h can be operably engaged with an optic, such as lens, directly or indirectly, as disclosed and shown in the various accommodating intraocular optic assemblies shown in FIGS. 1-8. The exemplary stanchion 12h includes an outer sleeve 46h defining a through-aperture 48h. The exemplary stanchion 12h also includes at least one inner member 50h positioned within the through-aperture 48h.

The exemplary through-aperture 48h has a first cross-sectional profile shape that is circular and constant along the length of the stanchion 12h. The inner member 50f has a second cross-sectional profile shape that is different than the first cross-sectional profile shape. The exemplary inner member 50f has a second cross-sectional profile shape that is elliptical. In operation, the outer sleeve 46h can be laterally compressed and elastically deformed when wound around an optic for insertion in the eye. This compressed state is shown in FIG. 11A and the exemplary inner member 50f is generally “floating” in the through-aperture 48h. When the accommodating intraocular optic assembly that includes the stanchion 12h is released in the eye, the outer sleeve 46h can elastically recover. The exemplary through-aperture 48h and the exemplary inner member 50f are sized such that the exemplary inner member 50f will then extend between and contact opposite sides of the exemplary through-aperture 48h. The exemplary inner member 50f will then act as brace inhibiting bending in a plane referenced at 58h but generally not inhibit bending in a plane referenced at 158h. The exemplary planes 58h, 158h are normal to one another.

It is noted that one or more embodiments of the present disclosure could include an inner member having an elliptical cross-sectional profile shape along one or more portions of its length and also have a circular cross-sectional profile shape along one or more other portions of its length. In such embodiments, a diameter of the circular cross-sectional profile shape of the outer sleeve could be greater than a diameter of the circular cross-sectional profile shape of the inner member so that, where the circular profiles overlap, the stanchion could more easily bend in all planes. Alternatively, the diameter of the circular cross-sectional profile shape of the outer sleeve could be the same as the diameter of the circular cross-sectional profile shape of the inner member so that, where the circular profiles overlap, bending in all planes would be more inhibited.

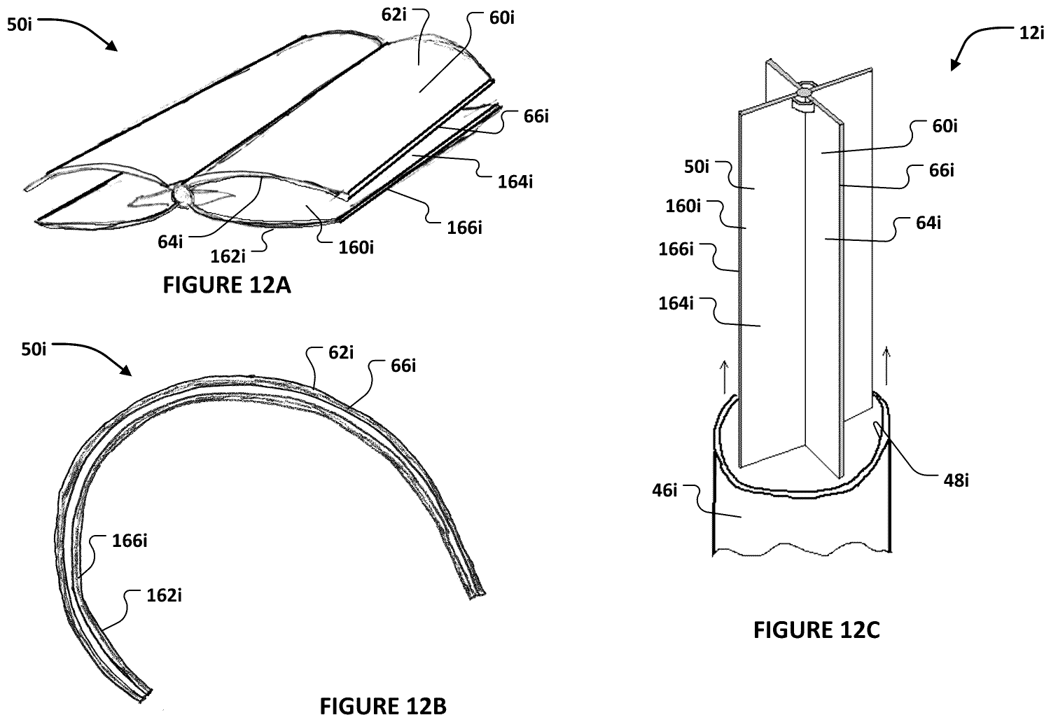

FIGS. 12A, 12B, and 12C are views of a fourth embodiment of an improvement in accommodating intraocular optic assemblies in the form of a portion of a stanchion 12i configured to provide selective, post implant stiffening. Any of the accommodating intraocular optic assemblies disclosed herein as well as any accommodating intraocular optic assemblies not disclosed herein can incorporate the stanchion 12i. The stanchion 12i can extend a length between a base end and a distal end. It is noted that FIGS. 12A-12C show portions of the stanchion 12i between the base and distal ends. The distal end of the stanchion 12i can be operably engaged with an optic, such as lens, directly or indirectly, as disclosed and shown in the various accommodating intraocular optic assemblies shown in FIGS. 1-8. The exemplary stanchion 12i includes an outer sleeve 46i defining a through-aperture 48i. The exemplary stanchion 12i also includes at least one inner member 50i positioned within the through-aperture 48i. FIG. 12C is an exploded view showing the components for assembly.

The exemplary inner member 50i comprises a flexible cruciate cross section that is defined by cruciate leaves, such as referenced by 60i, 160i. The cruciate leaf 60i includes a first surface 62i, a second surface 64i, and an edge 66i. The cruciate leaf 160i includes a first surface 162i, a second surface 164i, and an edge 166i. The cruciate leaves 60i, 160i are elastically foldable as shown in FIGS. 12A and 12B, whereby the inner member 50i is flattenable. It is noted that the inner member 50i can be utilized in one or more embodiments without an outer sleeve. In the operation of the exemplary embodiment, shown in FIGS. 12A-12C, the stanchion 12i can be flattened, resulting in the inner member 50i taking the form shown in FIG. 12A while in the outer sleeve 46i. Next, the stanchion 12i can be wound around an optic, resulting in the inner member 50i taking the form shown in FIG. 12B while in the outer sleeve 46i. When the accommodating intraocular optic assembly that includes the stanchion 12i is released in the eye, the inner member 50i and outer sleeve 46i can elastically recover, resulting in the inner member 50i taking the form shown in FIG. 12C. The exemplary inner member 12i will thus present resistance to lateral bending of the stanchion 12i. It is noted that in one or more embodiments of the present disclosure, a stanchion may include an outer sleeve with a plurality of inner members configured as the inner member 50i, wherein the inner members are positioned along the length of the stanchion at places where bending is not desired. At places along the length of the stanchion where bending is acceptable, the stanchion may only include a hollow outer sleeve.

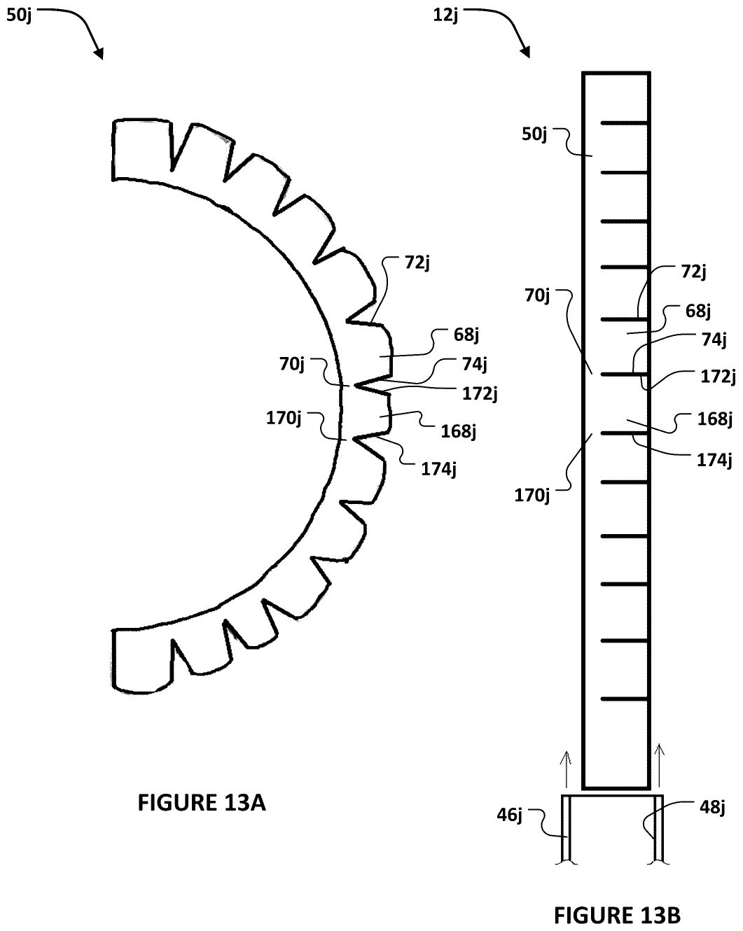

FIGS. 13A and 13B are side views of a fifth embodiment of an improvement in accommodating intraocular optic assemblies in the form of a stanchion 12j configured to provide selective, post implant stiffening. Any of the accommodating intraocular optic assemblies disclosed herein as well as any accommodating intraocular optic assemblies not disclosed herein can incorporate the stanchion 12j. The stanchion 12j can extend a length between a base end and a distal end. It is noted that FIGS. 13A and 13B show portions of the stanchion 12i between the base and distal ends. The distal end of the stanchion 12j can be operably engaged with an optic, such as lens, directly or indirectly, as disclosed and shown in the various accommodating intraocular optic assemblies shown in FIGS. 1-8. The exemplary stanchion 12j includes an outer sleeve 46j defining a through-aperture 48j. The exemplary stanchion 12j also includes at least one inner member 50j positioned within the through-aperture 48j.

The exemplary inner member 50j comprises a plurality of body segments, such as referenced at 68j and 168j, interconnected by webs, such as referenced at 70j and 170j. Each of said plurality of body segments includes opposite side surfaces, such as side surfaces 72j and 74j of the body segment 68j and side surfaces 172j and 174j of the body segment 168j. The side surfaces can contact one another when the inner member 50j is in a straight configuration, such as surfaces 74j and 172j, as shown in FIG. 13B. The side surfaces can be spaced from one another when the inner member 50j is bent into an arcuate configuration, as shown in FIG. 13A.

The inner member 50j can further comprise adhesive positioned on at least some of said opposite side surfaces. The adhesive would be a thin film on the surfaces and is therefore not referenced by number in the Figures. In one example, adhesive may be positioned on one or both of surfaces 74j and 172j such that the surfaces 74j, 172j will be fixedly adhered together when and subsequent to the inner member 50j being moved into a straight configuration. As set forth above, when the accommodating intraocular optic assembly that includes the stanchion 12j is released in the eye, the stanchion 12j can be wound around an optic and thus in the configuration shown in FIG. 13A. Upon release into the eye, the outer sleeve 46i can elastically recover and straighten to the extent possible. The accommodating intraocular optic assembly that includes the stanchion 12j can reach a steady-state configuration such that one or more adjacent pairs of body segments are straightened together and become adhered with the adhesive. Other adjacent pairs of body segments may remain at an angle relative to one another. It is also noted that the inner member 50j can be utilized in one or more embodiments without an outer sleeve.

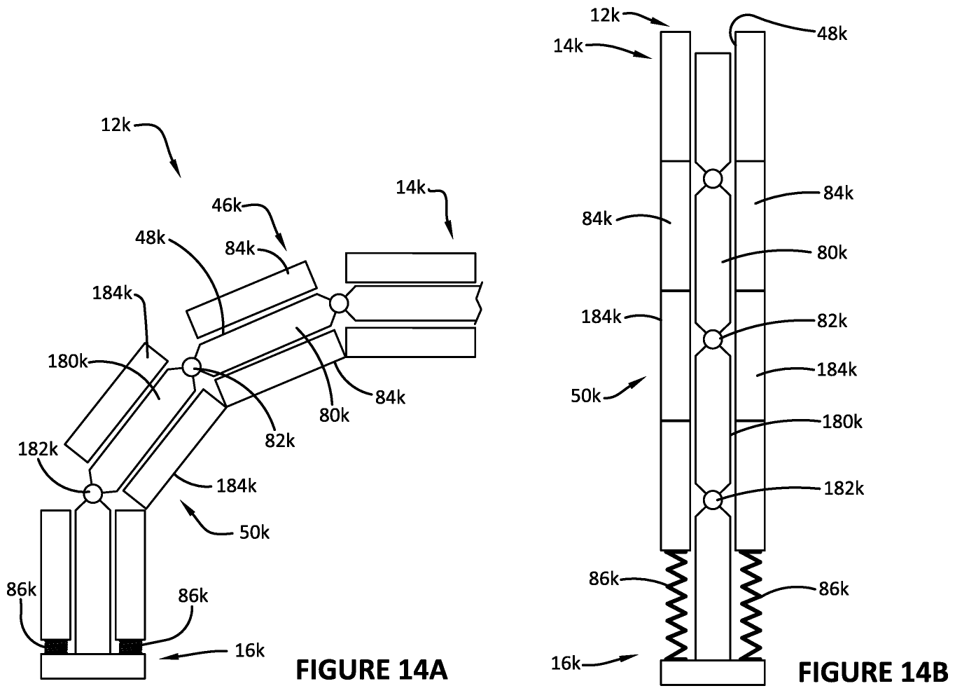

FIGS. 14A and 14B are side views of a sixth embodiment of an improvement in accommodating intraocular optic assemblies in the form of a stanchion 12k configured to provide selective, post implant stiffening. Any of the accommodating intraocular optic assemblies disclosed herein as well as any accommodating intraocular optic assemblies not disclosed herein can incorporate the stanchion 12k. The stanchion 12k can extend a length between a base end 14k and a distal end 16k. The distal end 16k can be operably engaged with an optic, such as lens, directly or indirectly, as disclosed and shown in the various accommodating intraocular optic assemblies shown in FIGS. 1-8. The exemplary stanchion 12k includes an outer sleeve 46k defining a through-aperture 48k. The exemplary stanchion 12k also includes at least one inner member 50k positioned within the through-aperture 48k.

The exemplary inner member 50k includes a plurality of links, such as links 80k and 180k, that are pivotally interconnected with one another. Exemplary pivot axes are referenced at 82k and 182k. The exemplary outer sleeve 46k further comprises a plurality of sleeve portions, such as sleeve portions 84k, 184k, connected to one another and moveable relative to one another. Adjacent sleeve portions can be interconnected to permit only relative rotational movement or both relative rotational movement and relative rectilinear movement. The exemplary stanchion 12k also includes a spring 86k disposed between the inner member 50k and the outer sleeve 46k.

In operation, when the accommodating intraocular optic assembly that includes the stanchion 12k is released in the eye, the stanchion 12k can be wound around an optic and thus in the configuration shown in FIG. 14A. Upon release into the eye, the spring 86k can elastically recover and can bias the outer sleeve away from the distal end 16k. As shown in FIG. 14A, when the stanchion 12k is in a bent or wound configuration, joints between sleeve portions are generally laterally aligned with the pivot axes. This alignment provides relatively minimal deterrence to movement among the links of the inner member 50k and the sleeve portions of the outer sleeve 46k and thus promotes straightening of the stanchion 12k. After the stanchion 12k has straightened, the joints between sleeve portions and the pivot axes are not laterally aligned. This lack of alignment deters movement among the links of the inner member 50k and the sleeve portions of the outer sleeve 46k and thus promotes the rigidity of the stanchion 12k. The stanchion 12k is thus stiffer after insertion in the eye.

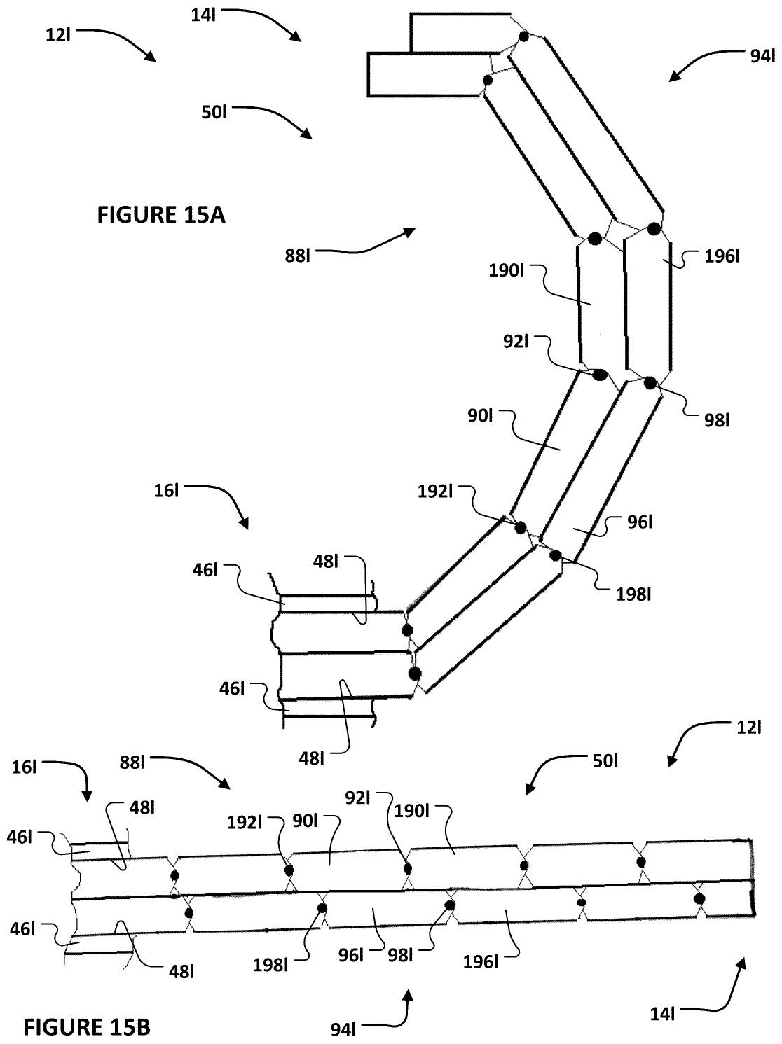

FIGS. 15A and 15B are side views of a seventh embodiment of an improvement in accommodating intraocular optic assemblies in the form of a stanchion 12l configured to provide selective, post implant stiffening. Any of the accommodating intraocular optic assemblies disclosed herein as well as any accommodating intraocular optic assemblies not disclosed herein can incorporate the stanchion 12l. The stanchion 12l can extend a length between a base end 14l and a distal end 16l. The distal end 16l can be operably engaged with an optic, such as lens, directly or indirectly, as disclosed and shown in the various accommodating intraocular optic assemblies shown in FIGS. 1-8. The exemplary stanchion 12l includes an outer sleeve 46l defining a through-aperture 48l. It is noted that the outer sleeve 46l is shown partially cut-off to enhance the clarity of the remaining structures in the Figures, but can extend the full length of the stanchion 12l. The exemplary stanchion 12l also includes at least one inner member 50l positioned within the through-aperture 48l.

The exemplary inner member 50l includes a first inner member 88l having a first plurality of links, such as links 90l and 190l, pivotally interconnected with one another. Exemplary pivot axes between links of the first plurality of links are referenced at 92l and 192l. The exemplary inner member 50l also includes a second inner member 94l having a second plurality of links, such as links 96l and 196l, pivotally interconnected with one another. Exemplary pivot axes between links of the second plurality of links are referenced at 98l and 198l. The first inner member 88l and the second inner member 94l are configured to slide across one another when the stanchion 12l bends in the first direction.

In operation, when the accommodating intraocular optic assembly that includes the stanchion 12l is released in the eye, the stanchion 12l can be wound around an optic and thus in the configuration shown in FIG. 15A. Upon release into the eye, the outer sleeve 46l can elastically recover and can bias the stanchion 12l into a straight configuration. As shown in FIG. 15A, when the stanchion 12l is in a bent or wound configuration, pivot axes between links of the first plurality of links are generally laterally aligned with the pivot axes between adjacent links of the second plurality of links. This alignment provides relatively minimal deterrence to movement among the links and thus promotes straightening of the stanchion 12l. After the stanchion 12l has straightened, the pivot axes between links of the first plurality of links are not laterally aligned with the pivot axes of the links of the second plurality of links. This lack of alignment deters movement among the links and thus promotes the rigidity of the stanchion 12l. The stanchion 12l is thus stiffer after insertion in the eye.

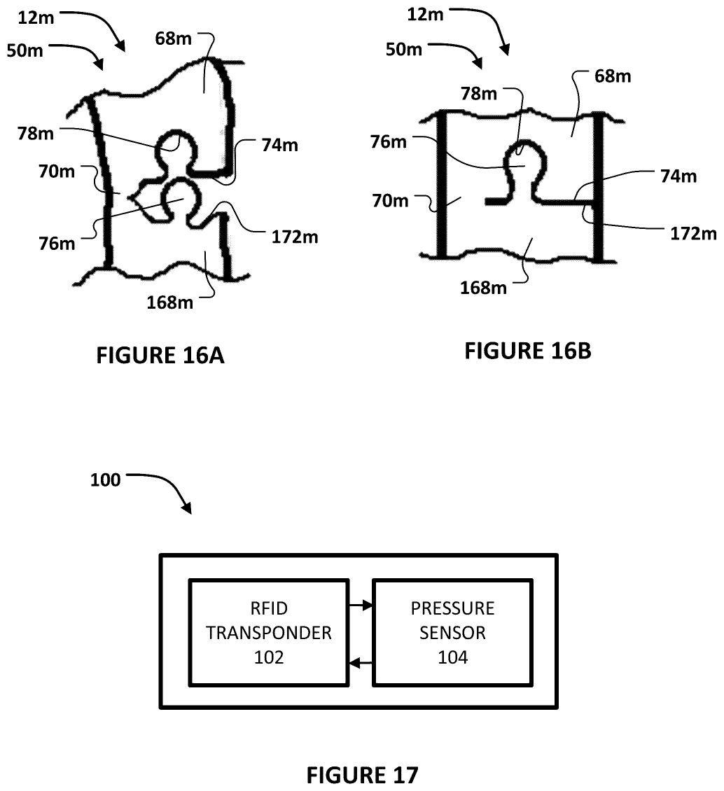

FIGS. 16A and 16B are side views of an eighth embodiment of an improvement in accommodating intraocular optic assemblies in the form of a portion of a stanchion 12m configured to provide selective, post implant stiffening. Any of the accommodating intraocular optic assemblies disclosed herein as well as any accommodating intraocular optic assemblies not disclosed herein can incorporate the stanchion 12m. The stanchion 12m can extend a length between a base end and a distal end. It is noted that FIGS. 16A and 16B show portions of the stanchion 12m between the base and distal ends. The distal end of the stanchion 12m can be operably engaged with an optic, such as lens, directly or indirectly, as disclosed and shown in the various accommodating intraocular optic assemblies shown in FIGS. 1-8. The exemplary stanchion 12m includes an outer sleeve defining a through-aperture. The exemplary stanchion 12m also includes at least one inner member 50m positioned within the through-aperture.

The embodiment shown in FIGS. 16A and 16B is substantially similar to the embodiment shown in FIGS. 13A and 13B. The exemplary inner member 50m comprises a plurality of body segments, such as referenced at 68m and 168m, interconnected by webs, such as referenced at 70m. Each of said plurality of body segments includes opposite side surfaces, such as side surface 74m of the body segment 68m and side surfaces 172m of the body segment 168m. The side surfaces can contact one another when the inner member 50m is in a straight configuration and can be spaced from one another when the inner member 50m is bent into an arcuate configuration. Rather than adhesive, the embodiment shown in FIGS. 16A and 16B further comprises one or more protuberances 76m on various side surfaces and apertures 78m configured to receive and mate with the protuberances 76m on adjacent side surfaces. It is noted that the inner member 50m can be utilized in one or more embodiments without an outer sleeve.

Another embodiment envisioned by the present disclosure is a stanchion having two components in a worm arrangement. One of the components could define a helical screw rod and the other component could define a threaded aperture receiving the helical screw rod. Selective rotation of helical screw rod during unflexing would allow the helical screw rod to rotate within the other component of the stanchion with little resistance. The inner surface of the other component could be formed to define different coefficients of friction in opposite rotational directions. As a result, rotation of the helical screw rod in one direction would be less inhibited than rotation of the helical screw rod in the opposite direction.

The present disclosure also provides embodiments in which materials can be selected for the stanchions whose mechanical properties alter after implantation due to the change in the in-vivo environment compared to the ex-vivo state. In one example, a stanchion can be made from material that becomes more rigid after implantation due to the change in temperature to body temperature. A possible material is Field's metal hybrid filler elastomer (FMHE) which can have a tunable stiffness, is non-toxic, and can be made safe for biological use. See phys.org/news/2023-01-smart-elastomer-self-tune-stiffness.html. Also, polyacrylic acid treated with calcium acetate produces a material that gets stiffer with increasing temperature. See newatlas.com/materials/heat-hardening-polyacrylic-hydrogel/.

Structural changes in response to temperature change could be an innate property of the material used to form the stanchion or could be realized through differential expansion of stanchion components resulting in locking of the stanchion in the un-flexed state. For example, fluid could be positioned within a stanchion and respond to temperature changes. Also, stanchions could be formed from different materials that have different rates of expansion/contraction in response to temperature change. Stiffness can also increase after implantation in the eye by choosing a material that hydrates upon exposure to the aqueous humor of the eye.

The present disclosure provides improvements in accommodating intraocular optic assemblies in the form of an intraocular pressure sensor assembly. FIG. 17 is a schematic view of a first intraocular pressure sensor assembly 100 configured to detect a level of pressure in the eye. The exemplary pressure sensor assembly 100 includes a radio frequency identification transponder 102 and a pressure sensor 104. The radio frequency identification transponder 102 is configured to generate a current in response to the presence of an electromagnetic transceiver field. Such fields are generated radio frequency identification “readers” or transceivers. The exemplary radio frequency identification transponder 102 is thus a passive transmitter.

The exemplary pressure sensor 104 is engaged with the exemplary radio frequency identification transponder 102. In response to the presence of an electromagnetic transceiver field, the exemplary radio frequency identification transponder 102 generate an electrical current and transmit the electrical current to the exemplary pressure sensor 104 and thereby provide electrical power to the exemplary pressure sensor 104. A pressure sensor that can utilized in embodiments of the present disclosure is sized 2.0×2.0×0.76 mm and can be found at st.com/en/mems-andsensors/lps22hb.html?icmp=pf261387_pron_pr_nov2014&sc=lps22hb-pr#documentation.

The exemplary pressure sensor 104, in response to receiving current from the exemplary radio frequency identification transponder 102, is configured to detect pressure within the eye and transmit a first signal corresponding to the detected pressure to the exemplary radio frequency identification transponder 104. The exemplary radio frequency identification transponder 104 is further configured to receive the first signal and transit the first signal outside of the eye. An exemplary placement for a pressure sensor assembly 100 is shown in FIG. 4.

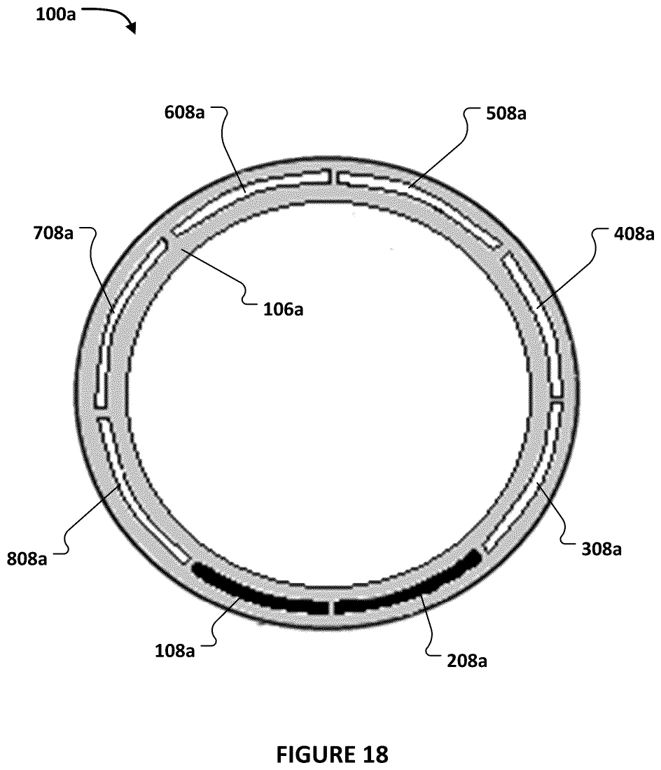

FIG. 18 is a front view of a second embodiment of an improvement in accommodating intraocular optic assemblies in the form of an intraocular pressure sensor 100a. The pressure sensor assembly 100a includes a plurality of cavities formed in an optic. The optic is referenced at 106a and can be a lens, a ring member, or the combination of a ring member and lens. The exemplary optic 106a is a ring member.

The cavities are referenced at 108a, 208a, 308a, 408a, 508a, 608a, 708a, and 808a. Each of the cavities can contain a fluid including a biocompatible dye vaporized within the gas. The biocompatible dye is configured to precipitate in response to changes in pressure.

The exemplary cavities 108a, 208a, 308a, 408a, 508a, 608a, and 708a and configured differently such that the biocompatible dye is each respective cavity precipitates at a different level of pressure. For example, cavity 108a can be configured such that the biocompatible dye precipitates at 5 mmHg, cavity 208a can be configured such that the biocompatible dye precipitates at greater than 5 mmHg and less than or equal to 10 mmHg, cavity 308a can be configured such that the biocompatible dye precipitates at greater than 10 mmHg and less than or equal to 15 mmHg, cavity 408a can be configured such that the biocompatible dye precipitates at greater than 15 mmHg and less than or equal to 20 mmHg, cavity 508a can be configured such that the biocompatible dye precipitates at greater than 20 mmHg and less than or equal to 25 mmHg, cavity 608a can be configured such that the biocompatible dye precipitates at greater than 25 mmHg and less than or equal to 30 mmHg, cavity 708a can be configured such that the biocompatible dye precipitates at greater than 30 mmHg and less than or equal to 35 mmHg, and cavity 808a can be configured such that the biocompatible dye precipitates at greater than 35 mmHg and less than or equal to 40 mmHg. In the example shown in FIG. 18, the pressure in the patient's eye is between 5-10 mmHg.

The pressure display can be visible to an examiner during slit-lamp exam after pupillary dilation, without pharmacological dilation using infrared imaging in scotopic (dark) conditions, and in any illumination if the device is part of an anterior chamber implant.

To form the cavities to display different levels of pressure, one embodiment could be cavities of varying size and/or thickness that are filled with a fluid such as perfluorocarbon gas which is biocompatible even if leaked, and hence often used in retinal surgery.

Alternatively, small changes in separation of the anterior and posterior surfaces of the cavity in response to pressure changes could be enhanced visually using phase shift biomicroscopy, or by displacement of a thin lining of fluid dye.

The use of any and all examples, or exemplary language (e.g., “such as”) provided herein is intended merely to be illustrative and does not pose a limitation on the scope of any invention disclosed herein unless otherwise claimed. As used herein, the singular forms “a,” “an,” and “the” include plural referents unless the context clearly dictates otherwise. Unless indicated otherwise by context, the term “or” is to be understood as an inclusive “or.” Terms such as “first”, “second”, “third”, etc. when used to describe multiple devices or elements, are so used only to convey the relative actions, positioning and/or functions of the separate devices, and do not necessitate either a specific order for such devices or elements, or any specific quantity or ranking of such devices or elements. Use of the terms “about” or “approximately” are intended to describe values above and/or below a stated value or range, as would be understood by one having ordinary skill in the art in the respective context.

It will be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof, unless indicated herein or otherwise clearly contradicted by context. Recitations of a value range herein, unless indicated otherwise, serves as a shorthand for referring individually to each separate value falling within the stated range, including the endpoints of the range, each separate value within the range, and all intermediate ranges subsumed by the overall range, with each incorporated into the specification as if individually recited herein. Unless indicated otherwise, or clearly contradicted by context, methods described herein can be performed with the individual steps executed in any suitable order, including: the precise order disclosed, without any intermediate steps or with one or more further steps interposed between the disclosed steps; with the disclosed steps performed in an order other than the exact order disclosed; with one or more steps performed simultaneously; and with one or more disclosed steps omitted.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to a particular embodiment disclosed herein as the best mode contemplated for carrying out this present disclosure, but that the present disclosure will be viewed as covering any embodiment falling within the scope of the appended claims. Also, the right to claim a particular sub-feature, sub-component, or sub-element of any disclosed embodiment, singularly or in one or more sub-combinations with any other sub-feature(s), sub-component(s), or sub-element(s), is hereby unconditionally reserved by the Applicant. Also, particular sub-feature(s), sub-component(s), and sub-element(s) of one embodiment that is disclosed herein can replace particular sub-features, sub-components, and sub-elements in another embodiment disclosed herein or can supplement and be added to other embodiments unless otherwise indicated by the drawings or this specification. Further, the use of the word “can” in this document is not an assertion that the subject preceding the word is unimportant or unnecessary or “not critical” relative to anything else in this document. The word “can” is used herein in a positive and affirming sense and no other motive should be presumed. More than one “invention” may be disclosed in the present disclosure; an “invention” is defined by the content of a patent claim and not by the content of a detailed description of an embodiment of an invention.

What is claimed is:

1. A accommodating intraocular optic assembly comprising:
an optic; and
at least one stanchion extending a length between a base end and a distal end, said distal end operably engaged with said optic one of directly and indirectly, and including:
an outer sleeve defining a through-aperture, and
at least one inner member positioned within said through-aperture.

2. The accommodating intraocular optic assembly of claim 1 wherein said at least one inner member further comprises rachet teeth.

3. The accommodating intraocular optic assembly of claim 2 wherein said at least one inner member further comprises:
a first elongate member defining a first set of rachet teeth; and
a second elongate member defining a second set of rachet teeth, wherein said first elongate member and said second elongate member are configured to slide across one another when said at least one stanchion bends in a first direction, wherein said first set of rachet teeth and said second set of rachet teeth are configured to slide across one another when said at least one stanchion bends in the first direction and are configured to lock together when said at least one stanchion bends in a second direction that is opposite to the first direction.

4. The accommodating intraocular optic assembly of claim 1 wherein said through-aperture has a first cross-sectional profile shape and said at least one inner member has a second cross-sectional profile shape that is different than said first cross-sectional profile shape.

5. The accommodating intraocular optic assembly of claim 4 wherein said first cross-sectional profile shape is constant along said length and circular and said second cross-sectional profile shape is elliptical.

6. The accommodating intraocular optic assembly of claim 1 wherein said at least one inner member comprises a flexible cruciate cross section with that cruciate leaves that are elastically foldable whereby said at least one inner member is flattenable.

7. The accommodating intraocular optic assembly of claim 1 wherein said at least one inner member comprises a plurality of body segments interconnected by webs.

8. The accommodating intraocular optic assembly of claim 7 wherein:
each of said plurality of body segments includes opposite side surfaces, said side surfaces contacting one another when said at least one inner member is in a straight configuration and spaced from one another when said at least one inner member is bent into an arcuate configuration; and
said at least one inner member further comprises adhesive positioned on at least some of said opposite side surfaces.

9. The accommodating intraocular optic assembly of claim 7 wherein:
each of said plurality of body segments includes opposite side surfaces, said side surfaces contacting one another when said at least one inner member is in a straight configuration and spaced from one another when said at least one inner member is bent into an arcuate configuration; and
said at least one inner member further comprises at least one protuberance and at least one aperture configured to mate with another and respectively defined in two of said opposite side surfaces.

10. The accommodating intraocular optic assembly of claim 1 wherein said at least one inner member further comprises:
a plurality of links pivotally interconnected with one another.

11. The accommodating intraocular optic assembly of claim 10 wherein said at least one inner member further comprises:
a first inner member having a first plurality of links pivotally interconnected with one another;
a second inner member having a second plurality of links pivotally interconnected with one another, said first inner member and said second inner member are configured to slide across one another when said at least one stanchion bends in the first direction.

12. The accommodating intraocular optic assembly of claim 10 wherein said outer sleeve further comprises:
a plurality of sleeve portions connected to one another and moveable relative to one another.

13. The accommodating intraocular optic assembly of claim 1 wherein said at least one stanchion further comprises:
a spring disposed between the said at least one inner member and said outer sleeve.

14. The accommodating intraocular optic assembly of claim 1 wherein said outer sleeve further comprises:
a plurality of sleeve portions connected to one another and moveable relative to one another.

15. A accommodating intraocular optic assembly comprising:
an optic; and
at least one stanchion extending a length between a base end and a distal end, said distal end operably engaged with said optic one of directly and indirectly, wherein said at least one stanchion is formed from a first material that becomes stiffer when inserted in the eye.

16. The accommodating intraocular optic assembly of claim 15 wherein said first material becomes stiffer when inserted in the eye because of body temperature.

17. The accommodating intraocular optic assembly of claim 15 wherein first material that becomes stiffer when inserted in the eye because of body temperature because of hydration of the stanchion from exposure to aqueous humor of the eye.

18. A accommodating intraocular optic assembly comprising:
an optic;
at least one stanchion extending a length between a base end and a distal end, said distal end operably engaged with said optic one of directly and indirectly; and
a pressure sensor assembly configured to detect a level of pressure in the eye.

19. The accommodating intraocular optic assembly of claim 18 wherein said pressure sensor assembly further comprises:
a radio frequency identification transponder; and
a pressure sensor engaged with said radio frequency identification transponder wherein:
said radio frequency identification transponder is configured, in response to the presence of an electromagnetic transceiver field to transmit electrical current to said pressure sensor and thereby provide electrical power to said pressure sensor, and
said pressure sensor, in response to receiving current from said radio frequency identification transponder, is configured to detect pressure within the eye and transmit a first signal corresponding to the detected pressure to said radio frequency identification transponder, wherein said radio frequency identification transponder is further configured to receive the first signal and transit the first signal outside of the eye.

20. The accommodating intraocular optic assembly of claim 18 wherein said pressure sensor assembly further comprises:
a plurality of cavities formed in said optic, each of said cavities containing fluid including a biocompatible dye vaporized within the gas, wherein said biocompatible dye is configured to precipitate in response to changes in pressure.
